(19) United States
(12) Patent Application Publication
(10) Pub. No.: US 2013/0287873 A1
(43) Pub. Date: Oct. 31, 2013

(54) PHARMACEUTICAL COMPOSITION AND METHOD FOR PREPARING A MEDICATION FOR PROMOTING WOUND HEALING

(71) Applicant: INDUSTRIAL TECHNOLOGY RESEARCH INSTITUTE, Hsinchu (TW)

(72) Inventors: Pei-Yi TSAI, Hsinchu City (TW); Hsiang-Wen TSENG, New Taipei City (TW); Yen-Chun CHEN, Hsinchu City (TW); Hui-Chun HSU, Hsinchu City (TW); Yi-Hung WEN, Hsinchu City (TW); Chun-Chung WANG, Kaohsiung City (TW); Shu-Jiau CHIOU, Hsinchu City (TW)

(73) Assignee: Industrial Technology Research Institute, Hsinchu (TW)

(21) Appl. No.: 13/871,853

(22) Filed: Apr. 26, 2013

(30) Foreign Application Priority Data
Apr. 27, 2012 (TW) 101115072
Dec. 26, 2012 (TW) 101150043

(51) Int. Cl. A61K 36/185; A61K 36/315

(52) U.S. Cl. CPC A61K 36/185; A61K 36/315
USPC 424/773; 424/725; 424/779; 424/774; 424/778; 424/777

(57) ABSTRACT

The disclosure provides a pharmaceutical composition for promoting wound healing, including an effective amount of a Sambucus plant or an Isatis plant as an active ingredient for promoting wound healing; and a pharmaceutically acceptable carrier or medium, wherein the Sambucus plant is Sambucus formosana Nakai, and the Isatis plant is Isatis indigotica Fort.

### CROSS REFERENCE TO RELATED APPLICATIONS

This application claims priority of Taiwan Patent Application No. 101150043, filed on Dec. 26, 2012, which claims priority of Taiwan Patent Application No. 101115072, filed on Apr. 27, 2012, the entireties of which are incorporated by reference herein.

### INCORPORATION BY REFERENCE OF SEQUENCE LISTING

A sequence listing submitted as a text file via EFS-Web is incorporated herein by reference. The text file containing the sequence listing is named “0965-A24043-US_Seq_Listing.txt”; its date of creation was Apr. 26, 2013; and its size is 5,575 bytes.

### TECHNICAL FIELD

The technical field relates to a pharmaceutical composition for promoting wound healing, and in particular relates to a pharmaceutical composition for promoting wound healing which uses a Sambucus plant as a main active ingredient, and can further comprise an Isatis plant as one of the active ingredients.

### BACKGROUND

Around the world, population are aging, and the incidence of people suffering wounds, and those wounds becoming infected, are also prevalent. Among the aged, wound infection and complications usually develop as chronic wounds, which can cause difficulty in their proper medical care and treatment. The reasons for the chronic nature of such wounds include diabetic ulcers/neuropathic ulcers, pressure/decubitus ulcers, arterial/venous ulcers, etc.

Diabetes patients are the main population in the world to whom chronic wounds occur. Using them as an example, 25% of the diabetes patients will suffer symptoms such as foot ulcers, difficulty in wound healing, proneness to infection, etc., and once the syndromes mentioned above occur, the amputation rate among diabetes patients is greater than 10%. Patients with chronic wounds during the healing process are subject to the problems of sustained ulcers and slow and difficult healing, the main reason for which is insufficient angiogenesis.

Sambucus formosana, classified in Rubiales order, Caprifoliaceae family, Sambucus genus, is an evergreen perennial herbaceous plant native to Taiwan. Sambucus. formosana has long been used as a folk herbal medicine in Taiwan to treat arthritis, jaundice, etc. The medicinal parts of Sambucus formosana comprise whole plants or roots. Recent studies showed that the extract of Sambucus formosana was capable of reducing the secretion of cytokines (such as IL-1α, IL-6, TNF-α, etc.), inhibiting maturation and growth of osteoclasts, and retarding bone loss such as osteopenia and osteoporosis.

Isatis indigotica, belonging to the Brassicaceae family, is a biennial herbaceous plant with bitter in taste and recognized as cold-nature in Chinese medicine. The roots of Isatis indigotica are the major parts for medicinal use. In traditional Chinese medicine, Isatis indigotica is recognized as a detoxicating, heat-clearing and anti-viral agent.

However, no report relates the Sambucus formosana or Isatis indigotica to angiogenesis.

### BRIEF SUMMARY

The disclosure provides a pharmaceutical composition for promoting wound healing, comprising: an effective amount of a Sambucus plant as an active ingredient for promoting wound healing; and a pharmaceutically acceptable carrier or medium.

The disclosure also provides a method for preparing a medication for promoting wound healing, comprising: using a Sambucus plant.

The disclosure provides another pharmaceutical composition for promoting wound healing, comprising: an effective amount of an Isatis plant as an active ingredient for promoting wound healing; and a pharmaceutically acceptable carrier or medium.

The disclosure further provides a method for preparing a medication for promoting wound healing, comprising: using an Isatis plant.

A detailed description is given in the following embodiments with reference to the accompanying drawings.

### BRIEF DESCRIPTION OF THE DRAWINGS

The embodiments of disclosure can be more fully understood by reading the subsequent detailed description and examples with references made to the accompanying drawings, wherein:

FIG. 1A shows the results of analyzing the proliferation of human umbilical vein endothelial cells treated with different single-plant extracts by MTT assay; p value<0.001: ***; p value<0.01:**; p value<0.05: *;

FIG. 1B shows the results of analyzing the proliferation of human umbilical vein endothelial cells treated with different single-plant extracts and plant extract mixture by MTT assay; p value<0.001: ***; p value<0.01: **; p value<0.05: *;

FIG. 1C shows the results of analyzing the proliferation of human umbilical vein endothelial cells treated with different extracts of different specific parts of the plant by MTT assay; p value<0.01: **; p value<0.05: *;

FIG. 2A shows photographs of fluorescence staining of the nucleuses of the human umbilical vein endothelial cells treated with different single-plant extracts;

FIG. 2B shows the results of counting human umbilical vein endothelial cells treated with different single-plant extracts; p value<0.001: ***; p value<0.01: **;

FIG. 2C shows photographs of fluorescence staining of nucleuses of the human umbilical vein endothelial cells treated with different single-plant extracts and a plant extract mixture;

FIG. 2D shows the results of counting human umbilical vein endothelial cells treated with different single-plant extracts and a plant extract mixture; p value<0.01: **; p value<0.05: *;

FIG. 3A shows fluorescence microscope photographs of tubes formed by the human umbilical vein endothelial cells treated with different extracts with different concentrations on the GFR-Metrigel;

FIG. 3B shows the results of quantitating the tube formation shown in FIG. 3A; p value<0.001: ***; p value<0.05: *;

FIG. 4A shows the results of a fluorescence analysis of the migration of the human umbilical vein endothelial cells treated with different extracts;

FIG. 4B shows the results of fluorescence quantitating the migration of the human umbilical vein endothelial cells treated with different extracts; p value<0.001:***; p value<0.01: **; p value<0.05: *;

FIG. 5A shows the results of a fluorescence analysis of the invasion of the human umbilical vein endothelial cells treated with different extracts;

FIG. 5B shows the results of fluorescence quantitating the invasion of the human umbilical vein endothelial cells treated with different extracts; p value<0.001: ***; p value<0.01: **; p value<0.05: *;

FIG. 6A shows microscope photographs of Matrigel taken from the subcutaneous tissue of mice treated with different plant extracts, obtained through H&E stain;

FIG. 6B shows the results of cell counting through H&E stain of Matrigel taken from the subcutaneous tissue of mice treated with different plant extracts; p value<0.01: ** ∘

FIG. 6C shows microscope photographs of Matrigel taken from the subcutaneous tissue of mice treated with different plant extracts, obtained through CD31 immunohistochemical stain;

FIG. 7A shows photographs of the animal experiment for healing diabetic wounds; Veh: no administration control group; TCM: the treatment group of equal ratio mixture of the extract of Sambucus formosana Nakai and the extracts of Isatis indigotica Fort.; and

FIG. 7B shows the percentage (%) of diabetic wound area in the animal experiment for healing diabetic wounds v.s. time; Veh: no administration control group; TCM: the treatment group of equal ratio mixture of the extract of Sambucus formosana Nakai and the extracts of Isatis indigotica Fort.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are shown schematically in order to simplify the drawing.

In one embodiment, the present disclosure provides a pharmaceutical composition for promoting wound healing which uses a Sambucus plant as a main active ingredient, wherein the Sambucus plant has the effects of promoting cell proliferation and angiogenesis, and is capable of promoting wound healing.

The pharmaceutical composition for promoting wound healing of the present disclosure mentioned above may comprise, but is not limited to, an effective amount of a Sambucus plant and a pharmaceutically acceptable carrier or medium, wherein the Sambucus plant is an active ingredient for promoting wound healing. Moreover, the Sambucus plant mentioned above has the effects of promoting cell proliferation and angiogenesis.

Examples of the Sambucus plant may comprise Sambucus formosana Nakai (also named Sambucus chinensis Lindl.), Sambucus japonica Thunb., Sambucus nigra L., Sambucus williamsii Hance or Sambucus williamsii Hance var. miquelii (Nakai) Y. C. Tang, etc., but are not limited thereto.

The Sambucus plant may only be subjected to a physical treatment of washing, cutting or grinding, etc. In this embodiment, a part of the Sambucus plant used in the pharmaceutical composition may comprise a whole plant of the Sambucus plant, a root of the Sambucus plant, a stem of the Sambucus plant, a leaf of the Sambucus plant, a flower of the Sambucus plant and/or a fruit of the Sambucus plant. Alternatively, the Sambucus plant may be subjected to an extraction process to yield an extract of the Sambucus plant. In this embodiment, a part of the Sambucus plant which is used in the pharmaceutical composition may comprise a whole plant of the Sambucus plant, a root of the Sambucus plant, a stem of the Sambucus plant, a leaf of the Sambucus plant, a flower of the Sambucus plant and/or a fruit of the Sambucus plant. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

The foregoing Sambucus plant may be a Sambucus formosana Nakai, and the Sambucus formosana Nakai which is identified by traditional plant taxonomy or gene taxonomy both can be the Sambucus formosana Nakai used in the disclosure. In one embodiment, the aforementioned Sambucus formosana Nakai may be a Sambucus formosana Nakai of which the sequence of an internal transcribed spacer of ribosomal DNA is a sequence with at least 60%, 70%, 80% or 90% sequence identity to SEQ ID NO: 1. In one specific embodiment, the aforementioned Sambucus formosana Nakai may be a Sambucus formosana Nakai of which the sequence of an internal transcribed spacer of ribosomal DNA is SEQ ID NO: 1 or a sequence with at least 80% sequence identity to SEQ ID NO: 1. In another embodiment, the aforementioned Sambucus formosana Nakai may be a Sambucus formosana Nakai of which the sequence of an intergenic non-coding region of chloroplast DNA, tmH-psbA, is a sequence with at least 60%, 70%, 80% or 90% sequence identity to SEQ ID NO: 5. In one specific embodiment, the aforementioned Sambucus formosana Nakai may be a Sambucus formosana Nakai of which the sequence of an intergenic non-coding region of chloroplast DNA, trnH-psbA, is SEQ ID NO: 5 or a sequence with at least 80% sequence identity to SEQ ID NO: 5.

In one embodiment, the Sambucus formosana Nakai mentioned above may only be subjected to a physical treatment of washing, cutting or grinding, etc. In this embodiment, a part of the Sambucus formosana Nakai used in the pharmaceutical composition may comprise a whole plant of the Sambucus formosana Nakai, a root of the Sambucus formosana Nakai, a stem of the Sambucus formosana Nakai, a leaf of the Sambucus formosana Nakai, a flower of the Sambucus formosana Nakai and/or a fruit of the Sambucus formosana Nakai, and in one specific embodiment, the part of the Sambucus formosana Nakai used in the pharmaceutical composition may be a whole plant of the Sambucus formosana Nakai.

In another embodiment, the Sambucus formosana Nakai mentioned above may be subjected to an extraction process to yield an extract of the Sambucus formosana Nakai. In this embodiment, a part of the Sambucus formosana Nakai used in the pharmaceutical composition may comprise a whole plant of the Sambucus formosana Nakai, a root of the Sambucus formosana Nakai, a stem of the Sambucus formosana Nakai, a leaf of the Sambucus formosana Nakai, a flower of the Sambucus formosana Nakai and/or a fruit of the Sambucus formosana Nakai, and in one specific embodiment, the part of the Sambucus formosana Nakai used in the pharmaceutical composition may be a whole plant of the Sambucus formosana Nakai. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

In another embodiment, the foregoing pharmaceutical composition for promoting wound healing of the present disclosure may further comprise an Isatis plant. In this embodiment, the foregoing pharmaceutical composition for promoting wound healing of the present disclosure comprises a Sambucus plant and an Isatis plant, a weight ratio of the Sambucus plant to the Isatis plant may be about 1-7:3-5, or about 1-5:3-5, but is not limited thereto. In addition, examples of the Isatis plant may comprise, but not limited to, Isatis indigotica Fort., Isatis oblongata DC., Isatis violascens, Isatis tinctoria L. var. praecox (Kit.) Koch, Isatis costata, Isatis minima Bunge and Isatis tinctoria L., etc.

The Isatis plant may only be subjected to a physical treatment of washing, cutting or grinding, etc. In this embodiment, a part of the Isatis plant used in the pharmaceutical composition may comprise a whole plant of the Isatis plant, a root of the Isatis plant, a stem of the Isatis plant, a leaf of the Isatis plant, a flower of the Isatis plant and/or a fruit of the Isatis plant.

Alternatively, the Isatis plant may be subjected to an extraction process to yield an extract of the Isatis plant. In this embodiment, a part of the Isatis plant used in the pharmaceutical composition may comprise a whole plant of the Isatis plant, a root of the Isatis plant, a stem of the Isatis plant, a leaf of the Isatis plant, a flower of the Isatis plant and/or a fruit of the Isatis plant. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

The foregoing Isatis plant may be an Isatis indigotica Fort., and the Isatis indigotica Fort. identified by traditional plant taxonomy or gene taxonomy both can be the Isatis indigotica Fort. used in the disclosure. In one embodiment, the aforementioned Isatis indigotica Fort. may be an Isatis indigotica Fort. of which the sequence of an internal transcribed spacer of ribosomal DNA is a sequence with at least 60%, 70%, 80% or 90% sequence identity to SEQ ID NO: 2. In one specific embodiment, the aforementioned Isatis indigotica Fort. may be an Isatis indigotica Fort. of which the sequence of an internal transcribed spacer of ribosomal DNA is SEQ ID NO: 2 or a sequence with at least 80% sequence identity to SEQ ID NO: 2. In another embodiment, the aforementioned Isatis indigotica Fort. may be an Isatis indigotica Fort. of which the sequence of an intergenic non-coding region of chloroplast DNA, tmF-trnL, is a sequence with at least 60%, 70%, 80% or 90% sequence identity to SEQ ID NO: 6. In one specific embodiment, the aforementioned Isatis indigotica Fort. may be an Isatis indigotica Fort. of which the sequence of an intergenic non-coding region of chloroplast DNA, trnF-trnL, is SEQ ID NO: 6 or a sequence with at least 80% sequence identity to SEQ ID NO: 6.

In one embodiment, the Isatis indigotica Fort. mentioned above may only be subjected to a physical treatment of washing, cutting or grinding, etc. In this embodiment, a part of the Isatis indigotica Fort. used in the pharmaceutical composition may comprise a whole plant of the Isatis indigotica Fort., a root of the Isatis indigotica Fort., a stem of the Isatis indigotica Fort. and/or a leaf of the Isatis indigotica Fort., and in one specific embodiment, the part of the Isatis indigotica Fort. used in the pharmaceutical composition may be a root of the Isatis indigotica Fort.

In another embodiment, the Isatis indigotica Fort. mentioned above may be subjected to an extraction process to yield an extract of the Isatis indigotica Fort. In this embodiment, a part of the Isatis indigotica Fort. used in the pharmaceutical composition may comprise a whole plant of the Isatis indigotica Fort., a root of the Isatis indigotica Fort., a stem of the Isatis indigotica Fort. and/or a leaf of the Isatis indigotica Fort., and in one specific embodiment, the part of the Isatis indigotica Fort. used in the pharmaceutical composition may be a root of the Isatis indigotica Fort. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

In one embodiment, the foregoing pharmaceutical composition for promoting wound healing of the present disclosure may comprise a Sambucus plant and an Isatis plant, wherein the Sambucus plant is a Sambucus formosana Nakai and the Isatis plant is an Isatis indigotica Fort., and the weight ratio of the Sambucus formosana Nakai and the Isatis indigotica Fort. may be about 1-7:3-5, or about 1-5:3-5, but is not limited thereto.

Since the pharmaceutical composition for promoting wound healing of the present disclosure has the effects of promoting cell proliferation and angiogenesis, it is capable of promoting wound healing. Cells which can be promoted to proliferate by using the pharmaceutical composition for promoting wound healing of the present disclosure may comprise human umbilical vein endothelial cells (HUVEC), but are not limited thereto. In addition, since the pharmaceutical composition for promoting wound healing of the present disclosure has the effects of promoting cell proliferation and angiogenesis, it can be applied as a treatment for the following symptoms or diseases. The symptoms or diseases may comprise a skin disease, coronary artery heart disease (CAHD), atherosclerosis, occlusion of cerebral arteries, ischemic heart disease, but are not limited thereto. Furthermore, the preceding skin disease may comprise, but is not limited to, a general trauma, a bedsore or a wound of a diabetes patient. Moreover, the pharmaceutical composition for promoting wound healing of the present disclosure can be further applied to medical cosmetology.

In one embodiment, the present disclosure also provides a method for preparing a medication for promoting wound healing, wherein the method may comprise using a Sambucus plant.

The Sambucus plant mentioned above may be any Sambucus plant without particular limitation, such as Sambucus formosana Nakai, Sambucus japonica Thunb., Sambucus nigra L., Sambucus williamsii Hance or Sambucus williamsii Hance var. miquelii (Nakai) Y. C. Tang, etc.

The Sambucus plant mentioned above may only be subjected to a physical treatment of washing, cutting or grinding, etc. In this embodiment, a part of the Sambucus plant used in the method may comprise a whole plant of the Sambucus plant, a root of the Sambucus plant, a stem of the Sambucus plant, a leaf of the Sambucus plant, a flower of the Sambucus plant and/or a fruit of the Sambucus plant.

Alternatively, the Sambucus plant mentioned above may be subjected to an extraction process to yield an extract of the Sambucus plant. In this embodiment, a part of the Sambucus plant used in the method may comprise a whole plant of the Sambucus plant, a root of the Sambucus plant, a stem of the Sambucus plant, a leaf of the Sambucus plant, a flower of the Sambucus plant and/or a fruit of the Sambucus plant. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

The foregoing Sambucus plant may be a Sambucus formosana Nakai, and the Sambucus formosana Nakai which is identified by traditional plant taxonomy or gene taxonomy both can be the Sambucus formosana Nakai used in the disclosure. In one embodiment, the aforementioned Sambucus formosana Nakai may be a Sambucus formosana Nakai of which the sequence of an internal transcribed spacer of ribosomal DNA is a sequence with at least 60%, 70%, 80% or 90% sequence identity to SEQ ID NO: 1. In one specific embodiment, the aforementioned Sambucus formosana Nakai may be a Sambucus formosana Nakai of which the sequence of an internal transcribed spacer of ribosomal DNA is SEQ ID NO: 1 or a sequence with at least 80% sequence identity to SEQ ID NO: 1. In another embodiment, the aforementioned Sambucus formosana Nakai may be a Sambucus formosana Nakai of which the sequence of an intergenic non-coding region of chloroplast DNA, trnH-psbA, is a sequence with at least 60%, 70%, 80% or 90% sequence identity to SEQ ID NO: 5. In one specific embodiment, the aforementioned Sambucus formosana Nakai may be a Sambucus formosana Nakai of which the sequence of an intergenic non-coding region of chloroplast DNA, trnH-psbA, is SEQ ID NO: 5 or a sequence with at least 80% sequence identity to SEQ ID NO: 5.

In one embodiment, the Sambucus formosana Nakai mentioned above may only be subjected to a physical treatment of washing, cutting or grinding, etc. In this embodiment, a part of the Sambucus formosana Nakai used in the method may comprise a whole plant of the Sambucus formosana Nakai, a root of the Sambucus formosana Nakai, a stem of the Sambucus formosana Nakai, a leaf of the Sambucus formosana Nakai, a flower of the Sambucus formosana Nakai and/or a fruit of the Sambucus formosana Nakai, and in one specific embodiment, the part of the Sambucus formosana Nakai used in the method may be a whole plant of the Sambucus formosana Nakai.

In another embodiment, the Sambucus formosana Nakai mentioned above may be subjected to an extraction process to yield an extract of the Sambucus formosana Nakai. In this embodiment, a part of the Sambucus formosana Nakai used in the method may comprise a whole plant of the Sambucus formosana Nakai, a root of the Sambucus formosana Nakai, a stem of the Sambucus formosana Nakai, a leaf of the Sambucus formosana Nakai, a flower of the Sambucus formosana Nakai and/or a fruit of the Sambucus formosana Nakai, and in one specific embodiment, the part of the Sambucus formosana Nakai used in the method may be a whole plant of the Sambucus formosana Nakai. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

In another embodiment, in the method for preparing a medication for promoting wound healing of the present disclosure, the Sambucus plant may be further used together with an Isatis plant. In this embodiment, at the condition of the Sambucus plant being used together with the Isatis plant, a weight ratio of the Sambucus plant to the Isatis plant may be about 1-7:3-5, or about 1-5:3-5, but not limited thereto.

Furthermore, the Isatis plant may be any Isatis plant without particular limitation, such as, Isatis indigotica Fort., Isatis oblongata DC., Isatis violascens, Isatis tinctoria L. var. praecox (Kit.) Koch, Isatis costata, Isatis minima Bunge or Isatis tinctoria L., etc.

The preceding Isatis plant may only be subjected to a physical treatment of washing, cutting or grinding, etc. In this embodiment, a part of the Isatis plant used in the method may comprise a whole plant of the Isatis plant, a root of the Isatis plant, a stem of the Isatis plant and/or a leaf of the Isatis plant, and in one specific embodiment, the part of the Isatis plant used in the method may be a root of the Isatis plant.

Alternatively, the preceding Isatis plant mentioned above may be subjected to an extraction process to yield an extract of the Isatis plant. In this embodiment, a part of the Isatis plant used in the method may comprise a whole plant of the Isatis plant, a root of the Isatis plant, a stem of the Isatis plant and/or a leaf of the Isatis plant, and in one specific embodiment, the part of the Isatis plant used in the method may be a root of the Isatis plant. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

The foregoing Isatis plant may be an Isatis indigotica Fort., and the Isatis indigotica Fort. identified by traditional plant taxonomy or gene taxonomy both can be the Isatis indigotica Fort. used in the disclosure. In one embodiment, the preceding Isatis indigotica Fort. may be an Isatis indigotica Fort. of which the sequence of an internal transcribed spacer of ribosomal DNA is a sequence with at least 60%, 70%, 80% or 90% sequence identity to SEQ ID NO: 2. In one specific embodiment, the aforementioned Isatis indigotica Fort. may be an Isatis indigotica Fort. of which the sequence of an internal transcribed spacer of ribosomal DNA is SEQ ID NO: 2 or a sequence with at least 80% sequence identity to SEQ ID NO: 2. In another embodiment, the aforementioned Isatis indigotica Fort. may be an Isatis indigotica Fort. of which the sequence of an intergenic non-coding region of chloroplast DNA, trnF-trnL, is a sequence with at least 60%, 70%, 80% or 90% sequence identity to SEQ ID NO: 6. In one specific embodiment, the aforementioned Isatis indigotica Fort. may be an Isatis indigotica Fort. of which the sequence of an intergenic non-coding region of chloroplast DNA, trnF-trnL, is SEQ ID NO: 6 or a sequence with at least 80% sequence identity to SEQ ID NO: 6.

In one embodiment, the Isatis indigotica Fort. mentioned above may only be subjected to a physical treatment of washing, cutting or grinding, etc. In this embodiment, a part of the Isatis indigotica Fort. used in the method may comprise a whole plant of the Isatis indigotica Fort., a root of the Isatis indigotica Fort., a stem of the Isatis indigotica Fort. and/or a leaf of the Isatis indigotica Fort., and in one specific embodiment, the part of the Isatis indigotica Fort. may be a root of the Isatis indigotica Fort.

In another embodiment, the Isatis indigotica Fort. mentioned above may be subjected to an extraction process to yield an extract of the Isatis indigotica Fort. In this embodiment, a part of the Isatis indigotica Fort. used in the method may comprise a whole plant of the Isatis indigotica Fort., a root of the Isatis indigotica Fort., a stem of the Isatis indigotica Fort. and/or a leaf of the Isatis indigotica Fort., and in one specific embodiment, the part of the Isatis indigotica Fort. used in the method may be a root of the Isatis indigotica Fort. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

In one embodiment, in the method for preparing a medication for promoting wound healing of the present disclosure, a Sambucus plant and an Isatis plant may be used together, wherein the Sambucus plant is Sambucus formosana Nakai, and the Isatis plant is Isatis indigotica Fort., and the weight ratio of the Sambucus plant and the Isatis plant may be about 1-7:3-5, or about 1-5:3-5.

In another embodiment, the present disclosure provides a pharmaceutical composition for promoting wound healing which uses an Isatis plant as a main active ingredient, wherein the Isatis plant has the effects of promoting cell proliferation and angiogenesis, and is capable of promoting wound healing.

The pharmaceutical composition for promoting wound healing of the present disclosure mentioned above may comprise, but is not limited to, an effective amount of an Isatis plant and a pharmaceutically acceptable carrier or medium, wherein the Isatis plant is an active ingredient for promoting wound healing. Moreover, the Isatis plant mentioned above has the effects of promoting cell proliferation and angiogenesis.

Examples of the Isatis plant may comprise Isatis indigotica Fort., Isatis oblongata DC., Isatis violascens, Isatis tinctoria L. var. praecox (Kit.) Koch, Isatis costata, Isatis minima Bunge and Isatis tinctoria L., etc., but are not limited thereto

The preceding Isatis plant may only be subjected to a physical treatment of washing, cutting or grinding, etc. In this embodiment, a part of the Isatis plant used in the pharmaceutical composition may comprise a whole plant of the Isatis plant, a root of the Isatis plant, a stem of the Isatis plant, a leaf of the Isatis plant, a flower of the Isatis plant and/or a fruit of the Isatis plant.

Alternatively, the preceding Isatis plant may be subjected to an extraction process to yield an extract of the Isatis plant. In this embodiment, a part of the Sambucus plant used in the pharmaceutical composition may comprise a whole plant of the Isatis plant, a root of the Isatis plant, a stem of the Isatis plant, a leaf of the Isatis plant, a flower of the Isatis plant and/or a fruit of the Isatis plant. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

The foregoing Isatis plant may be an Isatis indigotica Fort., and the Isatis indigotica Fort. identified by traditional plant taxonomy or gene taxonomy both can be the Isatis indigotica Fort. used in the disclosure. In one embodiment, the aforementioned Isatis indigotica Fort. may be an Isatis indigotica Fort. of which the sequence of an internal transcribed spacer of ribosomal DNA is a sequence with at least 60%, 70%, 80% or 90% sequence identity to SEQ ID NO: 2. In one specific embodiment, the aforementioned Isatis indigotica Fort. may be an Isatis indigotica Fort. of which the sequence of an internal transcribed spacer of ribosomal DNA is SEQ ID NO: 2 or a sequence with at least 80% sequence identity to SEQ ID NO: 2. In another embodiment, the aforementioned Isatis indigotica Fort. may be an Isatis indigotica Fort. of which the sequence of an intergenic non-coding region of chloroplast DNA, trnF-trnL, is a sequence with at least 60%, 70%, 80% or 90% sequence identity to SEQ ID NO: 6. In one specific embodiment, the aforementioned Isatis indigotica Fort. may be an Isatis indigotica Fort. of which the sequence of an intergenic non-coding region of chloroplast DNA, trnF-trnL, is SEQ ID NO: 6 or a sequence with at least 80% sequence identity to SEQ ID NO: 6.

In one embodiment, the foregoing Isatis indigotica Fort. may only be subjected to a physical treatment of washing, cutting or grinding, etc. In this embodiment, a part of the Isatis indigotica Fort. used in the pharmaceutical composition may comprise a whole plant of the Isatis indigotica Fort., a root of the Isatis indigotica Fort., a stem of the Isatis indigotica Fort. and/or a leaf of the Isatis indigotica Fort., and in one specific embodiment, the part of the Isatis indigotica Fort. used in the pharmaceutical composition may be a root of the Isatis indigotica Fort.

In another embodiment, the foregoing Isatis indigotica Fort. may be subjected to an extraction process to yield an extract of the Isatis indigotica Fort. In this embodiment, a part of the Isatis indigotica Fort. used in the pharmaceutical composition may comprise a whole plant of the Isatis indigotica Fort., a root of the Isatis indigotica Fort., a stem of the Isatis indigotica Fort. and/or a leaf of the Isatis indigotica Fort., and in one specific embodiment, the part of the Isatis indigotica Fort. used in the pharmaceutical composition may be a root of the Isatis indigotica Fort. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

Since the pharmaceutical composition for promoting wound healing of the present disclosure has the effects of promoting cell proliferation and angiogenesis, it is capable of promoting wound healing. Cells proliferation by using the pharmaceutical composition for promoting wound healing of the present disclosure may comprise human umbilical vein endothelial cells (HUVEC), but are not limited thereto. In addition, since the pharmaceutical composition for promoting wound healing of the present disclosure has the effects of promoting cell proliferation and angiogenesis, it can be applied as a treatment for the following symptoms or diseases. The symptoms or diseases may comprise a skin disease, coronary artery heart disease (CAHD), atherosclerosis, occlusion of cerebral arteries, ischemic heart disease, but are not limited thereto. Furthermore, the preceding skin disease may comprise, but is not limited to, a general trauma, a bedsore or a wound of a diabetes patient. Moreover, the pharmaceutical composition for promoting wound healing of the present disclosure can be further applied to medical cosmetology.

In one embodiment, the present disclosure also provides a method for preparing a medication for promoting wound healing, wherein the method may comprise using an Isatis plant.

The Isatis plant mentioned above may be any Isatis plant without particular limitation, such as, Isatis indigotica Fort., Isatis oblongata DC., Isatis violascens, Isatis tinctoria L. var. praecox (Kit.) Koch, Isatis costata, Isatis minima Bunge or Isatis tinctoria L., etc.

The preceding Isatis plant may only be subjected to a physical treatment of washing, cutting or grinding, etc. In this embodiment, a part of the Isatis plant used in the method may comprise a whole plant of the Isatis plant, a root of the Isatis plant, a stem of the Isatis plant and/or a leaf of the Isatis plant, and in one specific embodiment, the part of the Isatis plant used in the method may be a root of the Isatis plant.

Alternatively, the preceding Isatis plant mentioned above may be subjected to an extraction process to yield an extract of the Isatis plant. In this embodiment, a part of the Isatis plant used in the method may comprise a whole plant of the Isatis plant, a root of the Isatis plant, a stem of the Isatis plant and/or a leaf of the Isatis plant, and in one specific embodiment, the part of the Isatis plant used in the method may be a root of the Isatis plant. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

The foregoing Isatis plant may be an Isatis indigotica Fort., and the Isatis indigotica Fort. identified by traditional plant taxonomy or gene taxonomy both can be the Isatis indigotica Fort. used in the disclosure. In one embodiment, the preceding Isatis indigotica Fort. may be an Isatis indigotica Fort. of which the sequence of an internal transcribed spacer of ribosomal DNA is a sequence with at least 60%, 70%, 80% or 90% sequence identity to SEQ ID NO: 2. In one specific embodiment, the aforementioned Isatis indigotica Fort. may be an Isatis indigotica Fort. of which the sequence of an internal transcribed spacer of ribosomal DNA is SEQ ID NO: 2 or a sequence with at least 80% sequence identity to SEQ ID NO: 2. In another embodiment, the aforementioned Isatis indigotica Fort. may be an Isatis indigotica Fort. of which the sequence of an intergenic non-coding region of chloroplast DNA, trnF-trnL, is a sequence with at least 60%, 70%, 80% or 90% sequence identity to SEQ ID NO: 6. In one specific embodiment, the aforementioned Isatis indigotica Fort. may be an Isatis indigotica Fort. of which the sequence of an intergenic non-coding region of chloroplast DNA, trnF-trnL, is SEQ ID NO: 6 or a sequence with at least 80% sequence identity to SEQ ID NO: 6.

In one embodiment, the Isatis indigotica Fort. mentioned above may only be subjected to a physical treatment of washing, cutting or grinding, etc. In this embodiment, a part of the Isatis indigotica Fort. used in the method may comprise a whole plant of the Isatis indigotica Fort., a root of the Isatis indigotica Fort., a stem of the Isatis indigotica Fort. and/or a leaf of the Isatis indigotica Fort., and in one specific embodiment, the part of the Isatis indigotica Fort. used in the method may be a root of the Isatis indigotica Fort.

In another embodiment, the Isatis indigotica Fort. mentioned above may be subjected to an extraction process to yield an extract of the Isatis indigotica Fort. In this embodiment, a part of the Isatis indigotica Fort. used in the method may comprise a whole plant of the Isatis indigotica Fort., a root of the Isatis indigotica Fort., a stem of the Isatis indigotica Fort. and/or a leaf of the Isatis indigotica Fort., and in one specific embodiment, the part of the Isatis indigotica Fort. used in the method may be a root of the Isatis indigotica Fort. In one embodiment, the extraction process mentioned above does not have particular limitations, and it may be adjusted according to the properties of the plant and the environmental conditions. The extraction process may be a single step which is performed with a single solvent, or it may comprise various different extraction steps. In one embodiment, a solvent used in the extraction process mentioned above may comprise water, ethanol, methanol and/or another polar or non-polar solvents, etc., but it is not limited thereto.

Furthermore, in the pharmaceutical composition for promoting wound healing, the preceding pharmaceutically acceptable carrier may comprise, but is not limited to, a solvent, a dispersion medium, a coating, an antibacterial and antifungal agent, or an isotonic and absorption delaying agent. The pharmaceutical composition can be formulated into dosage forms for different administration routes utilizing conventional methods. For example, oral compositions can comprise, but are not limited to, tablets, capsules, emulsions, aqueous suspensions, dispersions and solutions.

In addition, for the external use of the pharmaceutical composition on skin, the pharmaceutically acceptable medium may act as a dilutant, dispersant or carrier for the active ingredient. The pharmaceutically acceptable medium may comprise materials commonly employed in skin care products such as water, liquid or solid emollients, silicone oils, emulsifiers, solvents, humectants, thickeners, powders, propellants and the like. The pharmaceutically acceptable medium can, in the absence of other adjuncts, form the balance of the compositions.

Moreover, the foregoing pharmaceutical composition for promoting wound healing of the present disclosure may be administered orally, parenterally, by an inhalation spray, via an implanted reservoir or by external use on skin.

The parenteral methods may comprise subcutaneous, intracutaneous, intravenous, intramuscular, intra-articular, intra-arterial, intrasynovial, intrasternal, intrathecal, and intralesional, as well as infusion techniques.

Manner for external use on skin may comprise directly applying the pharmaceutical composition of the present disclosure on a part of skin which needs to be treated or remedied.

Furthermore, all of the compositions mentioned may be manufactured in a form that can be spread on the skin, including, but not limited to, creams, ointments, gels, sprays, lotions, skin tonics, shampoos or mousses, etc. Skin sprays are generally composed of aerosolized copolymers, such as polyvinylpyrrolidone, vinyl acetate and the like, and may also function as a setting lotion. Skin gel preparations are similar to sprays in composition, but are in gel and alcohol-free form, and can coat the skin. A skin mousse is foam released under pressure from an aerosolized can. Skin creams may be a hydrophobic or hydrophilic cream, ointment, gel, emollient, spray, lotion, skin tonic, shampoo or mousse, suitably with additional ingredients suitable for use in skin cream of types known in the art, and such further ingredients can include petrolatum, waxes, lanolin, silicone, liposomes, vegetable, mineral oils, plasticizers, fragrances, preservatives, a penetration-enhancing agent, a pH adjusting agent or other suitable ingredients for skin creams. Such ingredients can moisturize skin, stabilize the active compound, increase the composition-skin contact, localize concentration and control the composition release.

Other specific ingredients which benefit skin, such as sunscreens, skin-lightening agents, and skin tanning agents may be also included in the compositions mentioned above. The medium may also further include adjuncts such as antioxidants, perfumes, opacifiers, preservatives, colourants, buffers, and the like.

### EXAMPLES

### Example 1

### Preparation of Plant Extract

1. Ethanol Extraction for Plant Material

200 g of powdered whole plant of Sambucus formosana Nakai, 200 g of powdered roots of Sambucus formosana Nakai, 200 g of powdered stems of Sambucus formosana Nakai and 200 g of powdered roots of Isatis indigotica Fort. were mixed with 5-fold volume of 95% ethanol, respectively, and subjected to a shaker extraction at 120 rpm for 7 days. The ethanol extraction solution was suction filtrated, then the filtrate was condensed to a volume of about 30 mL, and divided into portions into freeze-drying bottles to be freeze-dried, and the freeze-dried extracts obtained therefrom were weighted and record. The freeze-dried extracts were stored at 4° C. or −20° C.

### Example 2

### Identification of Plant Origin

1. DNA Extraction

The DNA of Sambucus formosana Nakai and Isatis indigotica Fort. was obtained according to the steps as follows:

(1) 500 mg to 1 g of dried plant material were ground to a fine powder in liquid nitrogen in a motor and pestle.

(2) After adding 5 mL of pre-warmed extraction buffer (100 mM Tris.HCl, pH 8.0, 20 mM EDTA, 1 M NaCl, 1% CTAB, 1% PVP-40), the mixture was incubated in a water bath at 65° C. for 20 min with gentle shaking.

(3) The sample solution was mixed with an equal volume of chloroform:isoamylalcohol (IAA) (24:1) and centrifuged at 11,000×g for 20 min at 4° C.

(4) The supernatant was transferred to new eppendorf tubes with two volumes of precipitation buffer (50 mM Tris.HCl, pH 8.0, 10 mM EDTA, 40 mM NaCl, 1% CTAB) and kept at room temperature for 1 hr then centrifuged at 11,000×g for 15 min at 4° C.

(5) The supernatant was carefully decanted and the pellet gently suspended in 350 μL of 1.2 M NaCl with 10 mg/mL RNase A.

(6) After incubation at 4° C. for 30 min, extraction with chloroform:IAA (24:1) was performed and the aqueous phase transferred to a new tube.

(7) One tenth volume of 3 M sodium acetate and 2 volumes of 95% ethanol were added to precipitate the DNA.

(8) After centrifugation at 12,000×g for 20 minutes, the DNA pellet was washed with 1 mL 70% ethanol, dried and dissolved in 50 to 100 μL TE buffer.

2. DNA Polymerase Chain Reaction (PCR)

The internal transcribed spacer of ribosomal DNA and an intergenic non-coding region of chloroplast DNA, trnH-psbA or trnF-trnL, were PCR amplified from a DNA sample of Sambucus formosana Nakai and a DNA sample of Isatis indigotica Fort obtained, respectively.

The contents of reaction mixture for polymerase chain reaction are shown in Table 1.

TABLE 1
The contents of reaction mixture for polymerase chain reaction

| Ingredients of reaction solution | Volume (ul) | Final concentration | |
|---|---|---|---|
| 10X reaction buffer | 5 | 1 | X |
| 10 mM dNTP | 1 | 0.25 | mM |
| 50 mM MgCl₂ | 2 | 2 | mM |
| DNA | 4 | | |
| Forward primer (10 pmol/uL) | 1 | 0.2 | pmol |
| Reverse primer (10 pmol/uL) | 1 | 0.2 | pmol |
| DNA polymerase (5 U/uL) | | 0.42 | U |

| | | |
|---|---|---|
| DMSO (4%) | optional | optional |

The primer sequences used for amplifying the internal transcribed spacer of ribosomal DNA of Sambucus formosana Nakai and Isatis indigotica Fort. are shown in Table 2.

TABLE 2
The primer sequences used for amplifying the internal
transcribed spacer of ribosomal DNA of Sambucus formosana
Nakai and Isatis indigotica Fort.

| | Sambucus formosana Nakai | Isatis indigotica Fort. |
|---|---|---|
| Forward primer | SEQ ID NO: 3 | SEQ ID NO: 3 |
| Reverse primer | SEQ ID NO: 4 | SEQ ID NO: 4 |

The primer sequences used for amplifying the intergenic non-coding region of chloroplast DNA of Sambucus formosana Nakai and Isatis indigotica Fort. are shown in Table 3.

TABLE 3
The primer sequences used for amplifying the intergenic
non-coding region of chloroplast DNA of Sambucus formosana
Nakai and Isatis indigotica Fort.

| | Sambucus formosana Nakai | Isatis indigotica Fort. |
|---|---|---|
| | (trnH-psbA) | (trnF-trnL) |
| Forward primer | SEQ ID NO: 7 | SEQ ID NO: 9 |
| Reverse primer | SEQ ID NO: 8 | SEQ ID NO: 10 |

The reaction mixture was placed in a PCR reaction tube, and water was added to a final volume of 50 μL.

The template DNA was denatured at 94° C. for 5 min and then subjected to 40 cycles of 94° C. for 30 sec, 56° C. for 30 sec, and 72° C. for 45 sec. The final cycle included an extension of 10 min at 72° C. The PCR products were examined with 1.5% agarose gel electrophoresis and purified for sequencing.

3. Sequencing

The purified fragment of the internal transcribed spacer of ribosomal DNA of Sambucus formosana Nakai and Isatis indigotica Fort. were sequenced, respectively. The sequence of the internal transcribed spacer of ribosomal DNA of Sambucus formosana Nakai was identified as SEQ ID NO: 1, and that of Isatis indigotica Fort. was identified as SEQ ID NO: 2.

Furthermore, the sequence of the intergenic non-coding region tmH-psbA of chloroplast DNA for Sambucus formosana Nakai was identified as SEQ ID NO: 5, and that of the intergenic non-coding region trnF-trnL of chloroplast DNA for Isatis indigotica Fort., was identified as SEQ ID NO: 6.

### Example 3

### Stimulate the Proliferation of Human Umbilical Vein Endothelial Cells (HUVEC) by Plant Extract

Screen for an Extract Promoting Proliferation of Human Umbilical Vein Endothelial Cells

A growth factor, VEGF, or notoginsenoside R1, etc. were used as control groups to screen a plant extract for promoting proliferation of vein endothelial cells and estimating activity of the plant extract. The experimental method is described as follows. Human umbilical vein endothelial cells were inoculated in a 96 well plate coated with gelatin with a density of 5×10³ cells/well, and cultured with M199 complete medium in a 37° C. incubator with 5% CO₂ overnight. The next day, the medium in the 96 well plate was changed to a medium containing a test plant extract with a specific concentration and a fresh 5% serum without ECGS, and then the culture plate was incubated. After 48 hours, activity of cells in the culture plate were analyzed by MTT assay and quantitated to obtain the information of the-functional specice of plant, the best part of the plant and the best extraction method for the plant for promoting the proliferation of human umbilical vein endothelial cells.

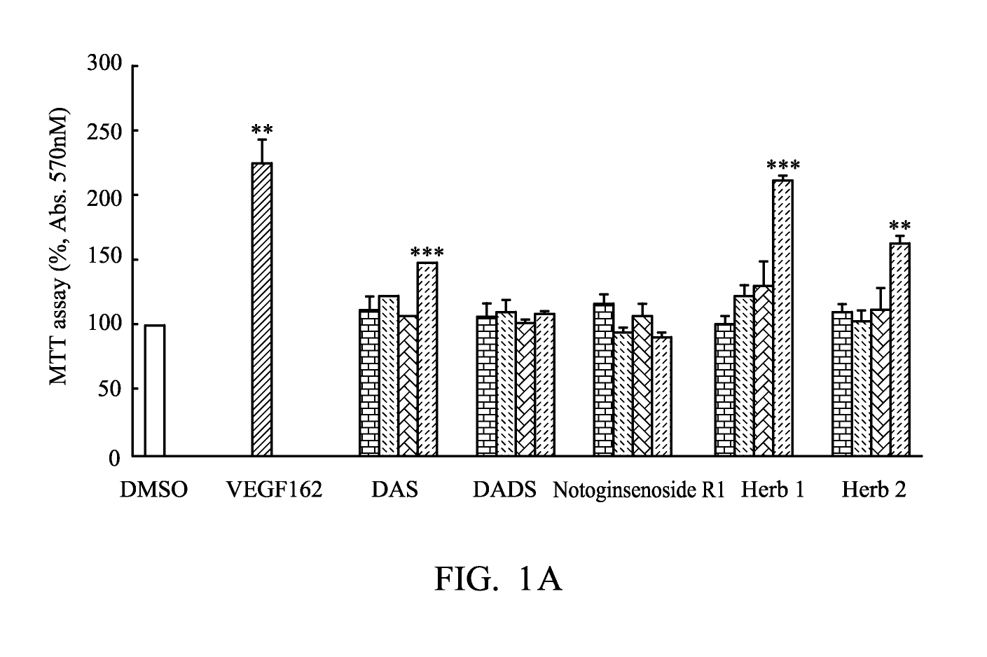

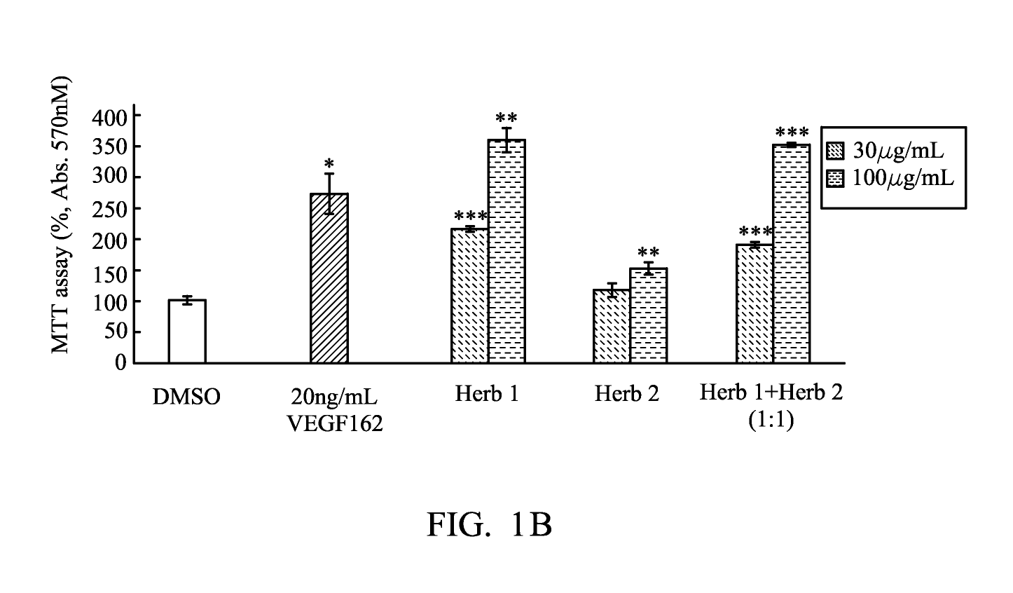

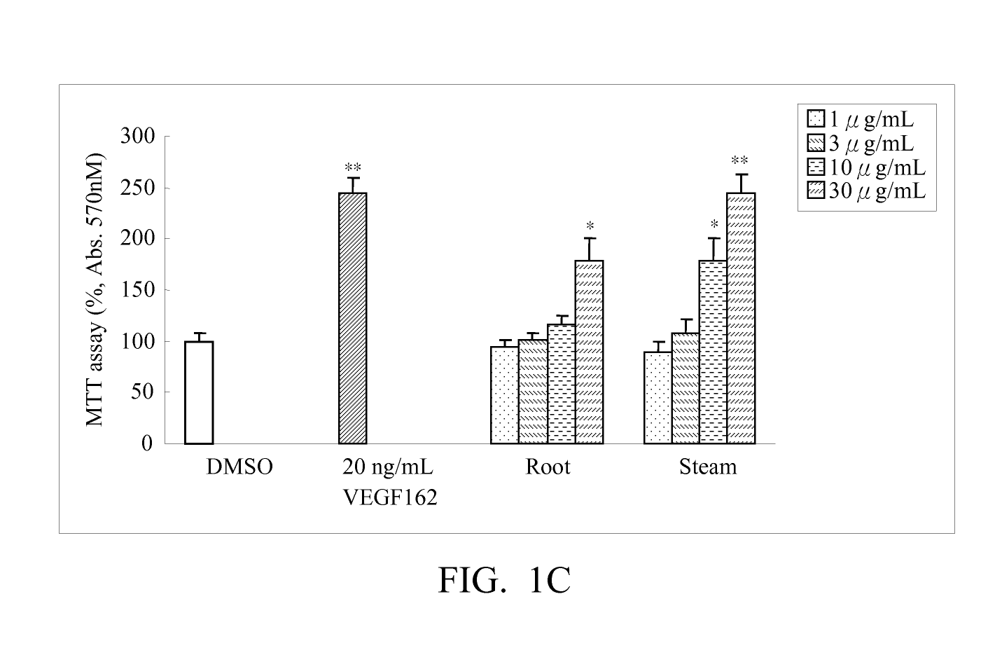

The extract of whole plants of Sambucus formosana Nakai (Herb 1) and the extract of roots of Isatis indigotica Fort. (Herb 2) were diluted 10-fold from 100 μg/mL which was an effective concentration from the primary screening to 0.1 μg/mL to determine the effective range and dose-response of the herbal extracts. The results of the proliferation assay for cells treated with different single-plant extracts are shown in FIG. 1A and Table 4, the results of the activity assay for cells treated with a combination of plant extracts are shown in FIG. 1B and Table 5, and the results of the proliferation assay for cells treated with extracts of specific parts of the plant are shown in FIG. 1C and Table 6. The two kinds of herbs with a concentration of 100 μg/mL have significant effects for promoting growth of vein endothelial cells, and are capable of achieving a promoting effect equivalent to that of VEGF.

TABLE 4
Effects of different single-plant extracts on vein endothelial cells

| | Treatment | | Cell growth rate (%) |
|---|---|---|---|
| Test Sample | concentration | | (mean ± standard deviation) |
| DMSO | — | | 100 ± 0 |
| VEGF₁₆₂ | 20 | ng/mL | 226 ± 17 |
| Diallyl sulfide (DAS) | 0.005 | μM | 113 ± 12 |
| | 0.05 | μM | 123 ± 11 |
| | 0.5 | μM | 108 ± 2 |
| | 5 | μM | 148 ± 1 |
| Diallyl disulfide (DADS) | 0.005 | μM | 107 ± 6 |
| | 0.05 | μM | 110 ± 2 |
| | 0.5 | μM | 101 ± 9 |
| | 5 | μM | 108 ± 4 |
| Notoginsenoside R1 | 0.05 | μg/mL | 118 ± 5 |
| | 0.5 | μg/mL | 95 ± 9 |
| | 5 | μg/mL | 107 ± 18 |
| | 50 | μg/mL | 91 ± 4 |
| Herb 1 | 0.1 | μg/mL | 102 ± 6 |
| | 1 | μg/mL | 123 ± 5 |
| | 10 | μg/mL | 131 ± 17 |
| | 100 | μg/mL | 213 ± 7 |
| Herb 2 | 0.1 | μg/mL | 111 ± 17 |
| | 1 | μg/mL | 104 ± 6 |
| | 10 | μg/mL | 112 ± 15 |
| | 100 | μg/mL | 163 ± 4 |

The extract of whole plants of Sambucus formosana Nakai and the extract of roots of Isatis indigotica Fort. were mixed with a ratio of 1:1 to from a mixture (containing 50 μg/mL of Herb 1 and 50 μg/mL of Herb 2), and the effect of the mixture on proliferation of vein endothelial cells was determined. The result is shown in FIG. 1B and Table 5. As shown in FIG. 1B and Table 5, when the positive control, 20 ng/mL VEGF₁₆₂ treatment group achieved a cell growth rate of 275±35%, at a condition of the treatment concentration being 30 μg/mL and 100 μg/mL, respectively, the treatment groups of extract of whole plants of Sambucus formosana Nakai achieved cell growth rates of 219±2% and 359±21%, the treatment groups of extract of roots of Isatis indigotica Fort. (Herb 2) achieved cell growth rates of 118±11% and 152±8%, and the treatment group of the mixture with a mixing ratio of 1:1 achieved cell growth rates of 192±1% and 351±2%. According to the above results, it is shown that although the concentrations of Herb 1 and Herb 2 are decreased to half in the mixture, the mixture could achieve the effect of promoting cell proliferation as only Herb 1 was used.

TABLE 5
Effects of different single-plant extracts and
plant extract mixture on vein endothelial cells

| | | |
|---|---|---|
| | | Cell growth rate (%) |
| Test Sample | Concentration | (mean ± standard deviation) |

| | | |
|---|---|---|
| DMSO | — | 100 ± 3 |

| | | | |
|---|---|---|---|
| VEGF₁₆₂ | 20 | μg/mL | 272 ± 35 |
| Herb 1 | 30 | μg/mL | 219 ± 2 |
| | 100 | μg/mL | 359 ± 21 |
| Herb 2 | 30 | μg/mL | 118 ± 11 |
| | 100 | μg/mL | 152 ± 8 |
| Herb 1 + | 30 | μg/mL | 192 ± 1 |
| Herb 2 (1:1) | 100 | μg/mL | 351 ± 2 |

The effects of extracts of different specific parts of the plants on vein endothelial cells were determined, and the results are shown in FIG. 1C and Table 6. The results show that extracts from roots or stems of Herb 1 all have effects of promoting growth of human umbilical vein endothelial cells. When the positive control, 20 ng/mL VEGF₁₆₂ treatment group, achieved a cell growth rate of 245±14%, the treatment group of 30 μg/mL of extract of roots of Sambucus formosana Nakai achieved a cell growth rate of 179±21%, and the treatment groups of 10 μg/mL and 30 μg/mL of extracts of stems of Sambucus formosana Nakai achieved cell growth rates of 178±23% and 244±18%, respectively. According to the above results, it can be known that extracts from roots or steams of Herb 1 all have the effect of promoting cell proliferation.

TABLE 6
Effects of extracts of different specific
parts of a plant on vein endothelial cells

| | | |
|---|---|---|
| | | Cell growth rate (%) |
| Test Sample | Concentration | (mean ± standard deviation) |

| | | | |
|---|---|---|---|
| DMSO | | | 100 ± 8 |
| VEGF₁₆₂ | 20 | ng/mL | 245 ± 14 |
| Herb 1-Root | 1 | μg/mL | 94 ± 7 |
| | 3 | μg/mL | 100 ± 8 |
| | 10 | μg/mL | 116 ± 10 |
| | 30 | μg/mL | 179 ± 21 |
| Herb 1-Stem | 1 | μg/mL | 90 ± 9 |
| | 3 | μg/mL | 108 ± 13 |
| | 10 | μg/mL | 178 ± 23 |
| | 30 | μg/mL | 244 ± 18 |

### Example 4

### Promotion of Proliferation of Human Umbilical Vein Endothelial Cells (HUVEC) by Plant Extract—Cell Counting Assay

Cell Counting for Human Umbilical Vein Endothelial Cells

Cells were treated and cultured for 48 hours according to the experimental method mentioned in Example 3. After that, the medium was washed out and the cells were fixed by 70% ethanol for 30 minutes. Then the fixing solution was removed, and the cells were stained with 0.5 μg/mL Hoechst 33258, and left to stand for 15 minutes in the dark. Afterwards, the dye was washed out and cell images were captured by an EVOS fluorescent microscope system, and the number of cell nuclei was counted under a 40× field of view to estimate the number of cells after treatment of theextracts.

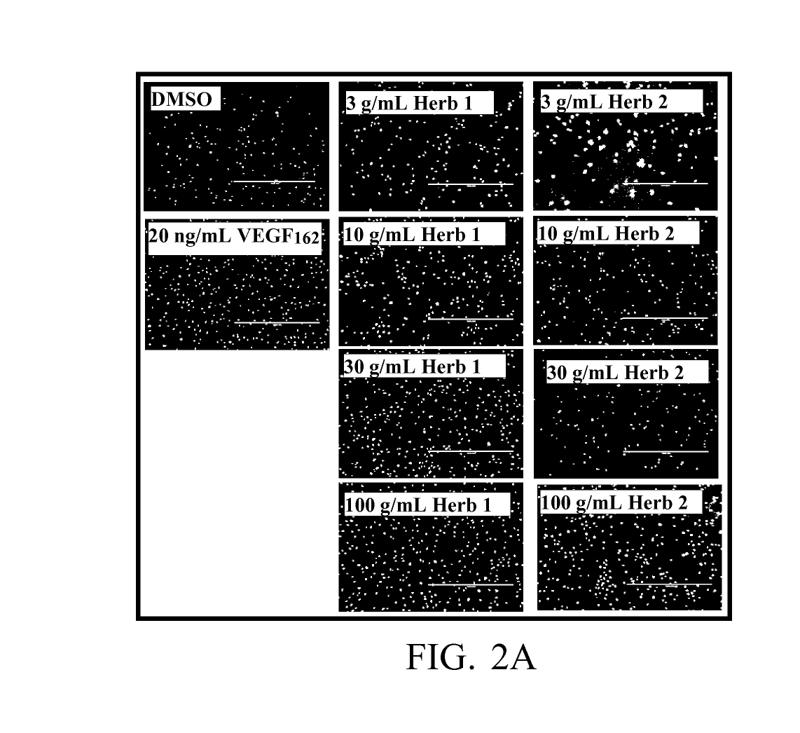

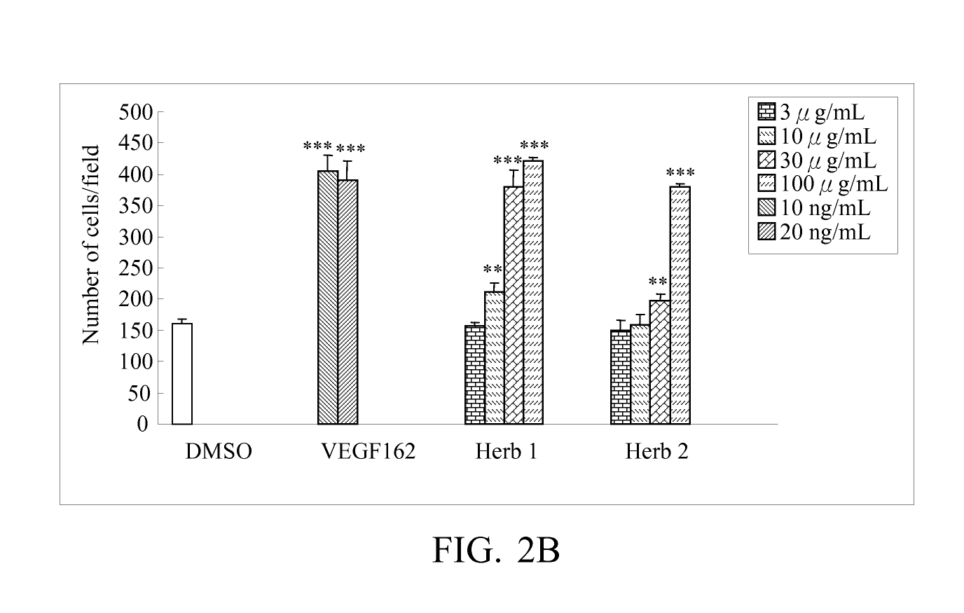

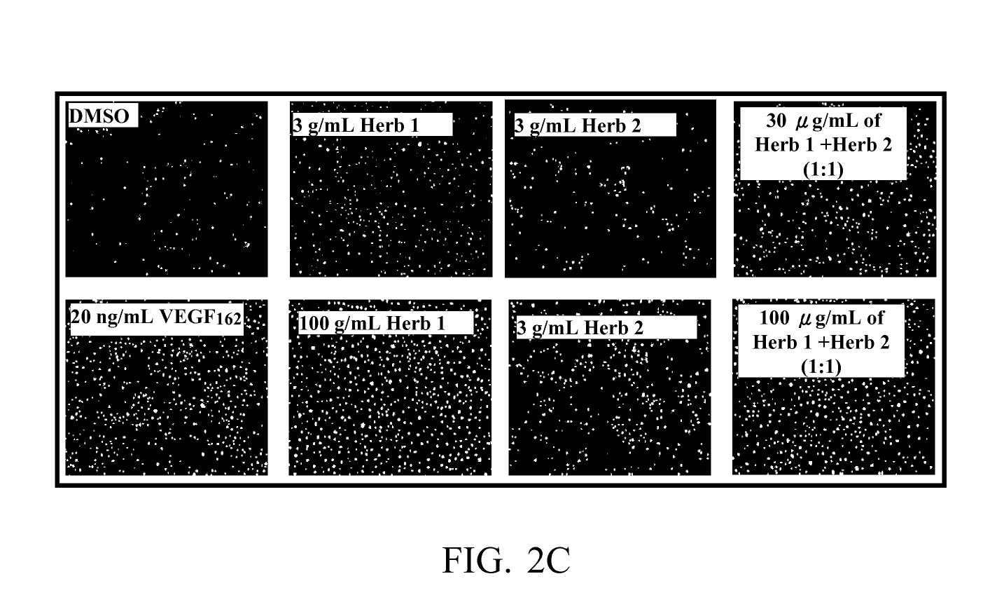

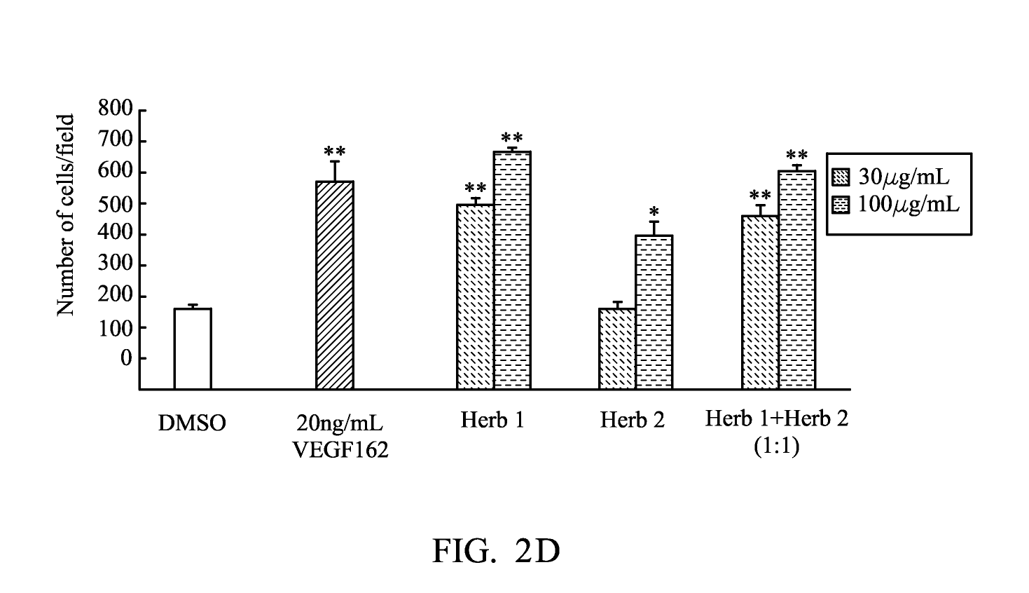

The images of the cells treated with different single-plant extracts at different concentrations were captured by the fluorescent microscope system, and the images which were captured are shown in FIG. 2A. FIG. 2B and Table 7 show the quantitative data of the cells shown in FIG. 2A. Referring to FIG. 2B and Table 7, the cell number of negative control, the DMSO treatment group, is 160±7, and the cell number of positive control, the ng/mL VEGF₁₆₂ treatment group, is 390±32. Under this analysis, as compared with negative control, the DMSO treatment group, the positive control, the 20 ng/mL VEGF₁₆₂ treatment group showed a significant difference. As compared with negative control, the DMSO treatment group, the positive control, the treatment groups of extracts of whole plants of Sambucus formosana Nakai (Herb 1) with concentrations equal to or greater than 10 μg/mL were capable of showing significant difference, and the cell numbers of the treatment groups of extracts of whole plants of Sambucus formosana Nakai (Herb 1) with concentrations of 3, 10, 30 and 100 μg/mL were 157±5, 211±15, 380±28 and 422±5, respectively, and that show a dose-response. In addition, the cell numbers of the treatment groups of the extracts of roots of Isatis indigotica Fort. (Herb 2) at concentrations of 3, 10, and 100 μg/mL were 150±16, 160±16, 197±11 and 380±5, respectively, and as compared with negative control, the treatment groups of extracts of roots of Isatis indigotica Fort. (Herb 2) with concentrations equal to or greater than 30 μg/mL were capable of showing significant difference.

TABLE 7
Effects of different plant extracts on
proliferation of vein endothelial cells

| | | |
|---|---|---|
| | | Cell number |
| Test Sample | Concentration | (mean ± standard deviation) |

| | | | |
|---|---|---|---|
| DMSO | | | 160 ± 7 |
| VEGF₁₆₂ | 20 | ng/mL | 390 ± 32 |
| Herb 1 | 3 | μg/mL | 157 ± 5 |
| | 10 | μg/mL | 211 ± 15 |
| | 30 | μg/mL | 380 ± 28 |
| | 100 | μg/mL | 422 ± 5 |
| Herb 2 | 3 | μg/mL | 150 ± 16 |
| | 10 | μg/mL | 160 ± 16 |
| | 30 | μg/mL | 197 ± 11 |
| | 100 | μg/mL | 380 ± 5 |

The extract of whole plants of Sambucus formosana Nakai and the extract of roots of Isatis indigotica Fort. were mixed at a ratio of 1:1 to from a mixture (containing 50 lag/mL of Herb 1 and 50 μg/mL of Herb 2), and the effect of the mixture on proliferation of vein endothelial cells was estimated.

The images of the cells treated with the 1:1 mixture of the extract of whole plants of Sambucus formosana Nakai and the extract of roots of Isatis indigotica Fort. and different single-plant extracts with different concentration were captured by the fluorescent microscope system, and shown in FIG. 2C. FIG. 2D and Table 8 show the cell counting results from FIG. 2C. Referring to FIG. 2D and Table 8, the cell number of negative control, the DMSO treatment group, is 163±23, and the cell number of positive control, the 20 ng/mL VEGF₁₆₂ treatment group, is 604±35. The cell numbers of the treatment groups of extracts of whole plants of Sambucus formosana Nakai (Herb 1) with concentrations of 30 μg/mL and 100 μg/mL were 503±49 and 673±28, respectively, and the cell numbers of the treatment groups of the extracts of roots of Isatis indigotica Fort. (Herb 2) with concentrations of 30 μg/mL and 100 μg/mL were 172±26 and 390±50, respectively. Furthermore, the cell numbers of the treatment groups of 1:1 mixture of the extract of whole plants of Sambucus formosana Nakai and the extract of roots of Isatis indigotica Fort. were capable of achieving 456±37 and 608±29, respectively, and the cell counting results mentioned above can confirm the results shown in FIG. 1B.

TABLE 8
Effects of the single-plant extracts and mixture of the
plant extract on proliferation of vein endothelial cells

| | | |
|---|---|---|
| | | Cell number |
| Test Sample | Concentration | (mean ± standard deviation) |

| | | | |
|---|---|---|---|
| DMSO | | | 163 ± 23 |
| VEGF₁₆₂ | 20 | ng/mL | 604 ± 49 |
| Herb 1 | 30 | μg/mL | 503 ± 28 |
| | 100 | μg/mL | 673 ± 10 |
| Herb 2 | 30 | μg/mL | 172 ± 26 |
| | 100 | μg/mL | 390 ± 50 |
| Herb 1 + | 30 | μg/mL | 456 ± 37 |
| Herb 2 (1:1) | 100 | μg/mL | 608 ± 29 |

### Example 5

### Promotion of Tube Formation of Human Umbilical Vein Endothelial Cells (HUVEC) by Plant Extract—Metrigel Based Tube Formation Assay (In Vitro Angiogenesis)

Assay for Tube Formation of Vein Endothelial Cells Assay (Tube Formation Assay)

Metrigel (or GFR-Metrigel (no growth factor)) was placed in a 4° C. refrigerator to unfreeze, then the Metrigel was mixed and diluted with M199 medium with a ratio 1:1 on ice, and added into a 48 well plate with a density of 100 μg/cm². The 48 well plate was placed in a 37° C. incubator to render the Metrigel into gel form. Human umbilical vein endothelial cells were suspended in M199 medium and adjusted to a concentration of 2×10⁵ cells/mL. After the cell suspension was mixed with a plant extract at a concentration which has been determined, 0.3 mL of cell suspension was added to the Metrigel-coated 48 well and then placed in a 37° C. incubator to be cultured for 3 hours. After that, cell images were captured by an EVOS microscope system, and the total length of the formed tubes under a fixed field of view was calculated by Image J software to obtain information regarding tube formation.

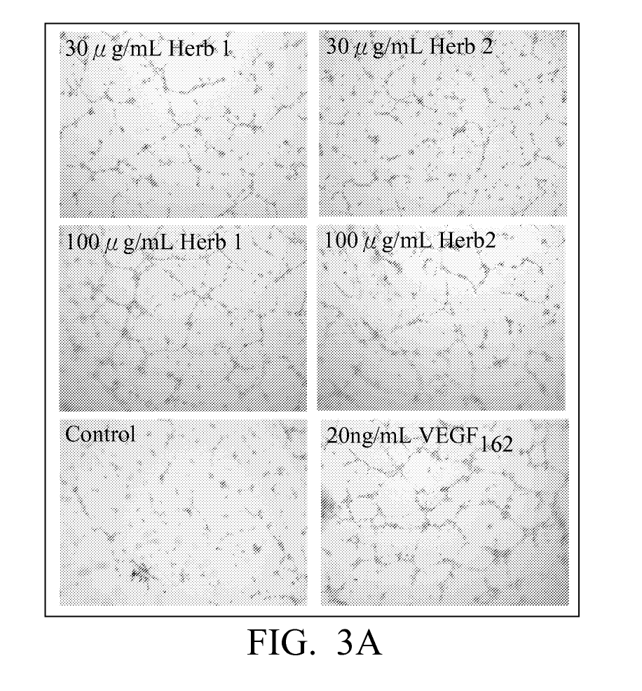

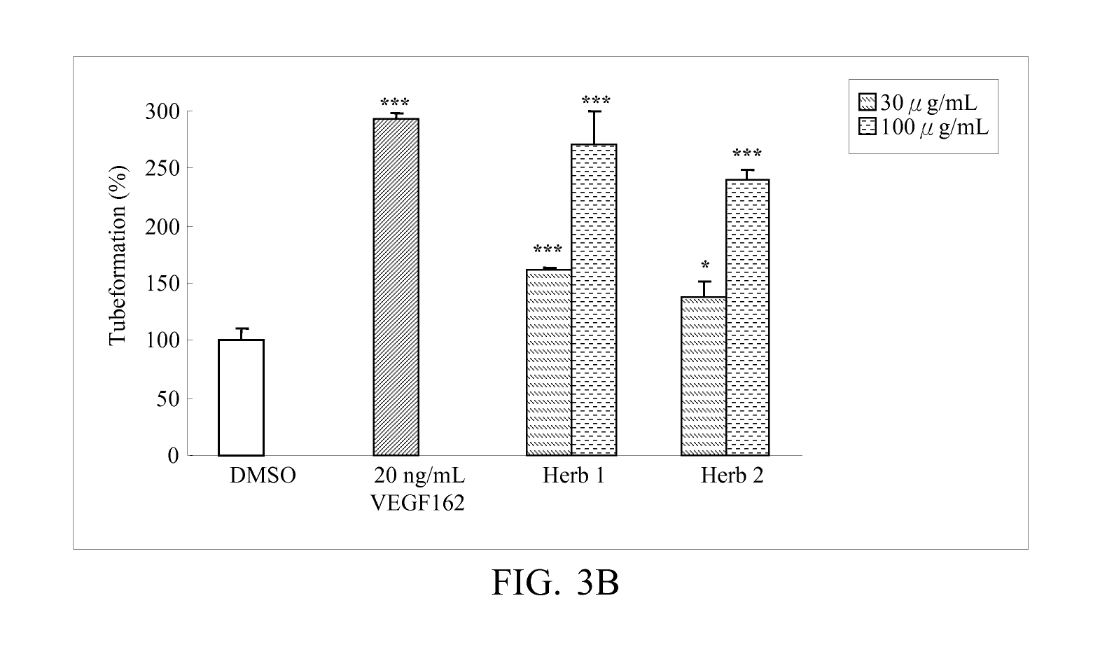

Tube formation was tested on GFR-Metrigel (no growth factor), and the results are shown in FIG. 3A. FIG. 3B and Table 9 show the results from quantitating the tube formation shown in FIG. 3A. As the tube length of the negative control, DMSO treatment group, is considered as 100±11%, the tube length of the positive control, the 20 ng/mL VEGF₁₆₂ treatment group, reached 294±5%, the tube length of the treatment groups of extracts of whole plants of Sambucus formosana Nakai (Herb 1) with concentrations of 30 μg/mL and 100 μg/mL reached 163±2% and 271±29%, respectively, and under the same concentrations, the tube length of the treatment groups of extracts of roots of Isatis indigotica Fort. (Herb 2) reached 138±13% and 241±9%, respectively. The two kinds of herbal extract both have promoting effects for tube formation, and show dose-responses.

TABLE 9
Effect of different single-plant extracts
on tube formation of vein endothelial cells

| | | |
|---|---|---|
| | | Tube length ± |
| Sample | Concentration | standard deviation (%) |

| | | | |
|---|---|---|---|
| DMSO | | | 100 ± 11 |
| VEGF₁₆₂ | 20 | ng/mL | 294 ± 5 |
| Herb 1 | 30 | μg/mL | 163 ± 2 |
| | 100 | μg/mL | 271 ± 29 |
| Herb 2 | 30 | μg/mL | 138 ± 13 |
| | 100 | μg/mL | 241 ± 9 |

### Example 6

### Promotion of Cell Migration of Human Umbilical Vein Endothelial Cells (HUVEC) by Plant Extract

The Effects of Different Plant Extracts on Cell Migration of Vein Endothelial Cells Were Analyzed by Cell Migration Assay.

Human umbilical vein endothelial cells were inoculated to an Oris™ Cell Migration Assay Collagen I plate with a density of 2×10⁴ cells/well, and cultured overnight until the cells attached to the bottom of the plate. The cells were changed to be cultured with M199 medium containing 2% serum for 6 hours. After that, the Oris™ Stoppers of the plate were removed by an Oris™ Stopper Tool, and a plant extract with a specific concentration were added to the plate. After 18 hours, the cells were stained with Calcein AM fluorescence dye, cell migration regions were marked with Oris™ Detection Mask, and cell images were captured by an EVOS fluorescent microscope system. Finally, cells were fixed with 10% formaldehyde for 15 minutes. After the fixing solution was removed, the nucleuses of the cells were stained with 0.5 μg/mL of Hoechst 33258, and then the cell number under a fixed field of view was calculated by Image J software.

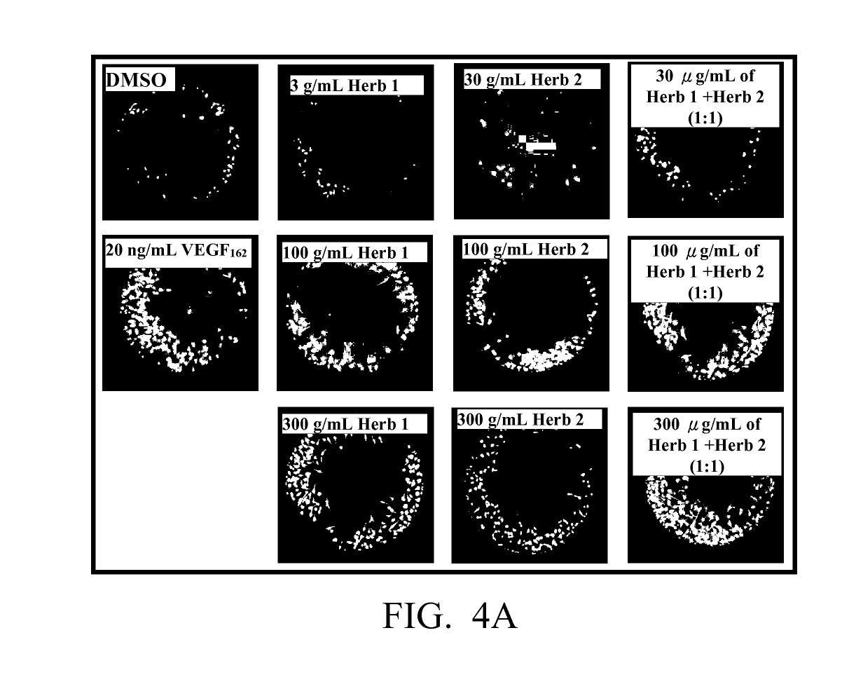

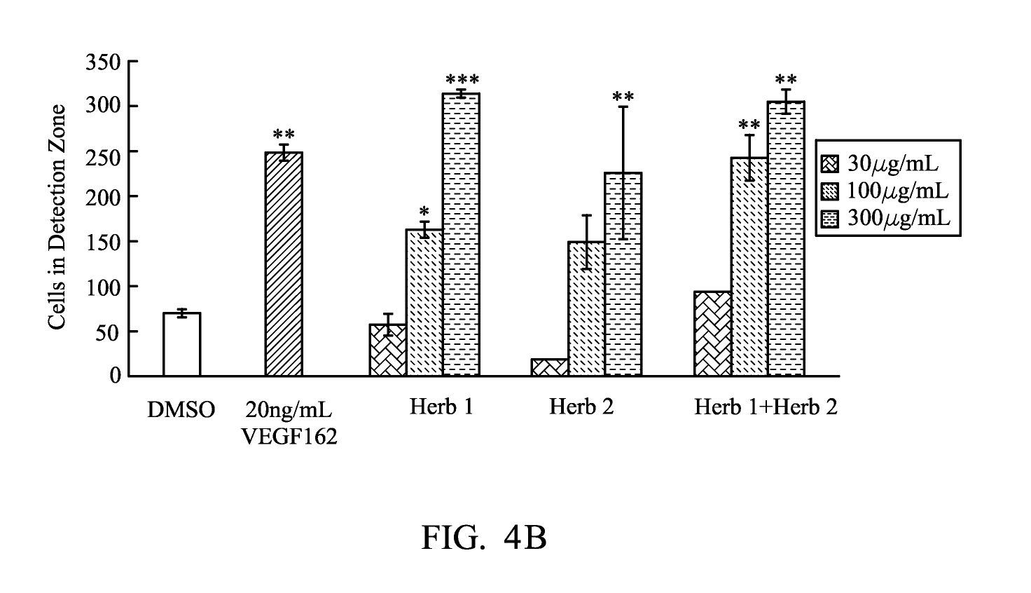

The image of the results of cell migration tested by The Oris™ Cell Migration Assay are shown in FIG. 4A, and FIG. 4B and Table 10 show the results from quantitating the numbers of migrating cells shown in FIG. 4A. As the number of migrating cells of the negative control, DMSO treatment group, is 71±10, the number of migrating cells of the positive control, the 20 ng/mL VEGF₁₆₂ treatment group, reached 251±6, the number of migrating cells of the treatment groups of extracts of whole plants of Sambucus formosana Nakai (Herb 1) with concentrations of 30 μg/mL, 100 μg/mL and 300 μg/mL were 60±13, 159±9 and 307±3, respectively, and the number of migrating cells of the treatment groups of extracts of roots of Isatis indigotica Fort. (Herb 2) with concentrations of 30 μg/mL, 100 μg/mL and 300 μg/mL were 22±0, 148±28 and 234±72, respectively. In addition, the number of migrating cells of the treatment groups of 1:1 mixture of the extract of whole plants of Sambucus formosana Nakai (Herb 1) and the extract of roots of Isatis indigotica Fort. (Herb 2) with concentrations of 30 μg/mL, 100 μg/mL and 300 μg/mL reached 93±0, 242±37 and 296±29, respectively. The results show that the two kinds of herbal extracts and the 1:1 mixture of the two extracts all have promoting effects for cell migration, and show dose-responses.

TABLE 10
Effects of different single-plant extracts and mixture of
the plant extract on migration of vein endothelial cells

| | | |
|---|---|---|
| | | Number of migrating |
| Test Sample | Concentration | cells ± standard deviation |

| | | | |
|---|---|---|---|
| DMSO | | | 71 ± 10 |
| VEGF₁₆₂ | 20 | ng/mL | 251 ± 6 |
| Herb 1 | 30 | μg/mL | 60 ± 13 |
| | 100 | μg/mL | 159 ± 9 |
| | 300 | μg/mL | 307 ± 3 |
| Herb 2 | 30 | μg/mL | 22 ± 0 |
| | 100 | μg/mL | 148 ± 28 |
| | 300 | μg/mL | 234 ± 72 |
| Herb 1 + | 30 | μg/mL | 93 ± 0 |
| Herb 2 | 100 | μg/mL | 242 ± 15 |
| | 300 | μg/mL | 296 ± 14 |

### Example 7

### Promotion of Cell Invasion of Human Umbilical Vein Endothelial Cells (HUVEC) by Plant Extract

The Effects of Different Plant Extracts on Cell Invasion of Vein Endothelial Cells Were Analyzed by Cell Invasion Assay.

500 μL of culture medium containing 5×10⁴ cells was mixed with plant extract with specific concentration and inculated onto a BioCoat™ Matrigel™ Growth factor reduced (GFR) invasion chamber. After 18 hours, the cells which stayed on the upper layer of the inserts of the chamber were wiped by a cotton swab, and the inserts of the chamber were rinsed with PBS buffer, and the cells that invaded to the lower layer of the inserts were stained with Calcein AM fluorescence dye, and cell images were captured by an EVOS microscope system. Next, cells were fixed with 10% formaldehyde for 15 minutes. Afterward, the fixing solution was removed, the cell nuclei were stained with 0.5 μg/mL of Hoechst 33258, and then the cell number under a fixed field of view was calculated by Image J software.

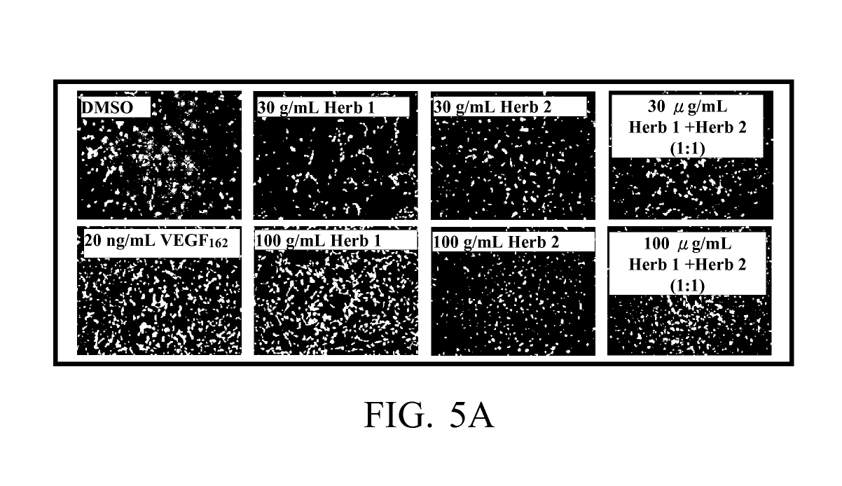

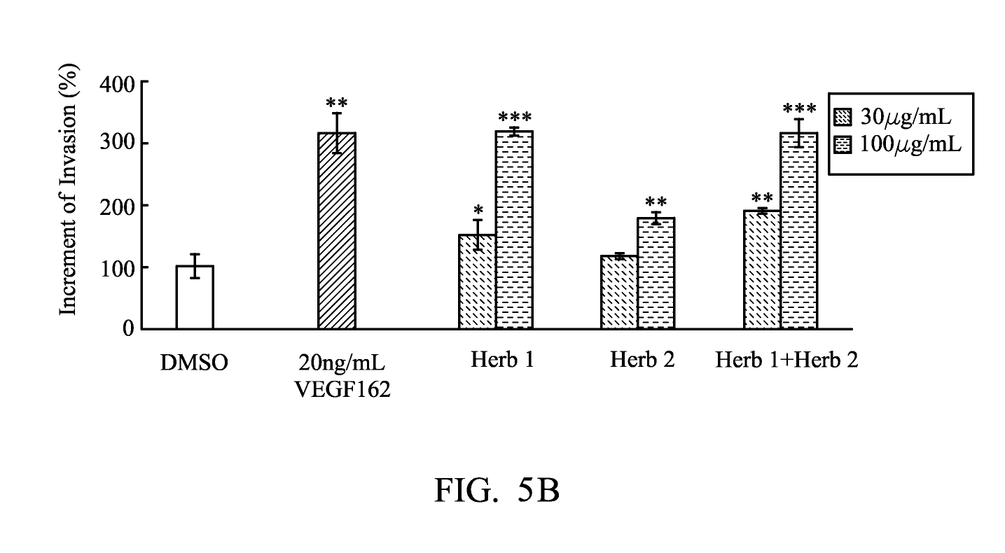

The imaging results from invasion of cells promoted by plant extracts analyzed by BioCoat™ Matrigel™ Growth factor reduced (GFR) invasion chamber are shown in FIG. 5A, and FIG. 5B and Table 11 show the results from quantitating the numbers of invading cells shown in FIG. 5A with percentages. As the cell invasion rate of the negative control, DMSO treatment group, is considered as 100±17%, the cell invasion rate of the positive control, the 20 ng/mL VEGF₁₆₂ treatment group, reached 310±36%, the cell invasion rate of the treatment groups of extracts of whole plants of Sambucus formosana Nakai (Herb 1) with concentrations of 30 μg/mL and 100 μg/mL were 49±23% and 313±4%, respectively, and the cell invasion rate of the treatment groups of extracts of roots of Isatis indigotica Fort. (Herb 2) with concentrations of 30 μg/mL and 100 μg/mL were 118±2% and 176±5%, respectively. In addition, the cell invasion rate of the treatment groups of 1:1 mixture of the extract of whole plants of Sambucus formosana Nakai (Herb 1) and the extract of roots of Isatis indigotica Fort. (Herb 2) with concentrations of 30 μg/mL and 100 μg/mL reached 186±6% and 313±15%, respectively. The results show that the two kinds of herbal extract and the 1:1 mixture of the two extracts all have promoting effects for cell migration, and show dose-responses.

TABLE 11
Effect of different single-plant extracts and mixture of
the plant extract on invasion of vein endothelial cells

| | | |
|---|---|---|
| | | Invasion rate ± |
| Sample | Concentration | standard deviation (%) |

| | | | |
|---|---|---|---|
| DMSO | | | 100 ± 17 |
| VEGF₁₆₂ | 20 | ng/mL | 310 ± 36 |
| Herb 1 | 30 | μg/mL | 149 ± 23 |
| | 100 | μg/mL | 313 ± 4 |
| Herb 2 | 30 | μg/mL | 118 ± 2 |
| | 100 | μg/mL | 176 ± 5 |
| Herb 1 + | 30 | μg/mL | 186 ± 6 |
| Herb 2 | 100 | μg/mL | 313 ± 15 |

### Example 8

### Animal Experiment for Promoting Angiogenesis by Plant Extract

8.1 Method for Angiogenesis Experiment In Vivo

Matrigel is purchased from BD Biosciences and purified from Engelbreth-Holm-Swarm (EHS) mice sarcoma, which contains rich extracellular matrix and growth factors, is capable of polymerizing to a gel form matter at room temperature and 37° C., and is capable of mimicking the growth environment for vein endothelial cells and angiogenesis. Activities of the plant extracts mentioned above for promoting angiogenesis in living animals were tested. 1.5×10⁶ human umbilical vein endothelial cells were mixed with 250 μL of Matrigel. The mixture was mixed with different plant extracts and then implanted into the immunodeficiency mice (SCID mice) abdominals subcutaneously. After 14 days, the mice were sacrificed and the Matrigel plugs were taken for further analysis. Hemotoxylin & Eosin (H&E) stain was applied to detect cell morphology and immunohistochemistry analysis for newly formed vessel was performed to the Matrigel plugs. After the Matrigel plugs were taken, the Matrigel plugs were fixed with 4% formaldehyde for 24 hours. Then the Matrigel plugs were paraffin embedded and sections were made therefrom. Cell morphology was stained via Hemotoxylin & Eosin (H&E) staining and vein endothelial cells and angiogenesis were stained via anti-CD31 immunohistochemical stain. For cell proliferation, Results of H&E staining was considered as a result for observing cell proliferation. Each stained tissue section was observed by a microscope at 200× magnification, and 2 observed regions were selected randomly, and cell counting was performed on the observed regions. For angiogenesis, results of CD31 immunohistochemical staining were considered as results for observing angiogenesis. By CD31 immunohistochemical staining, whether a plant extract is capable of promoting proliferation and angiogenesis of human umbilical vein endothelial cells of the Matrigel plugs is observed.

8.2 Results for Angiogenesis Experiment in Living Animals

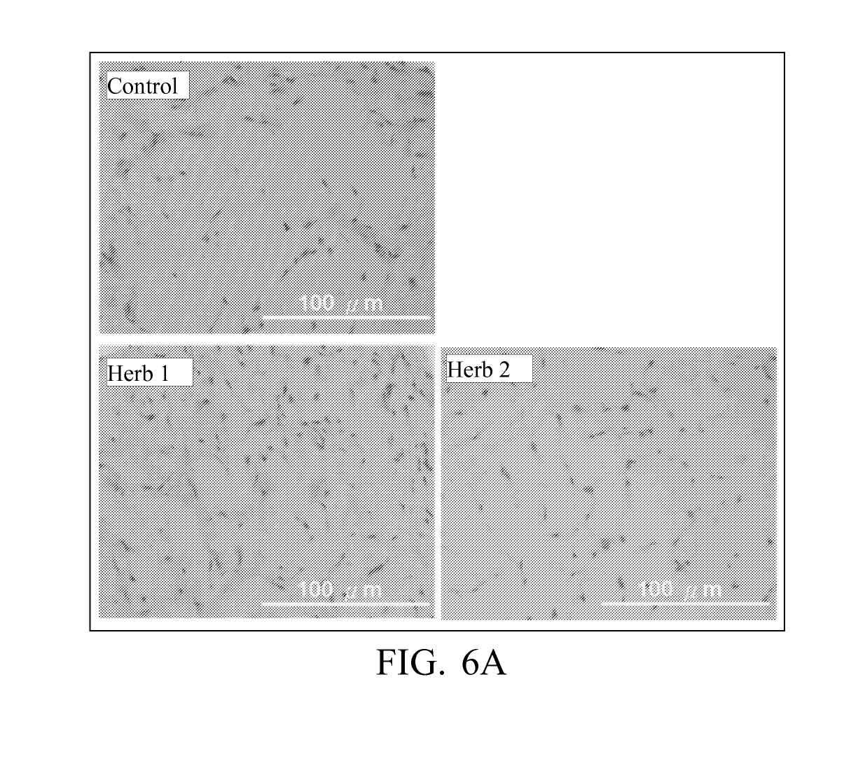

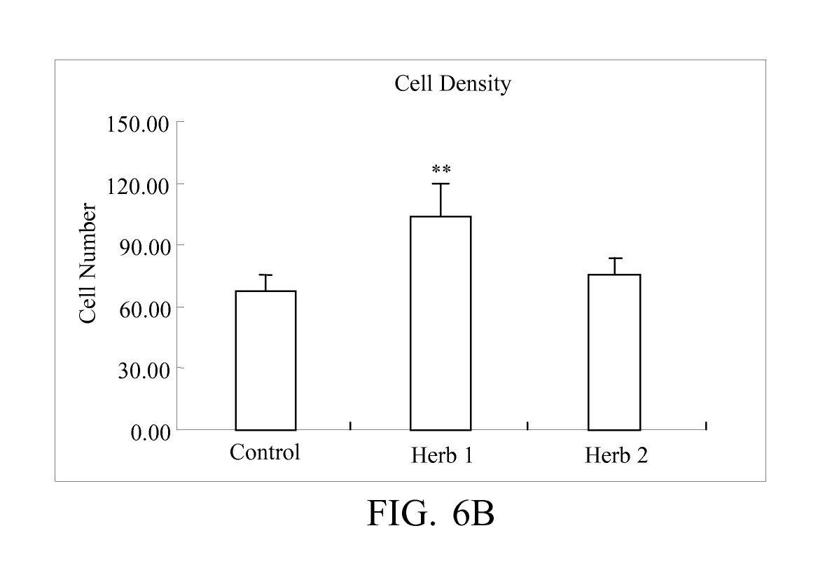

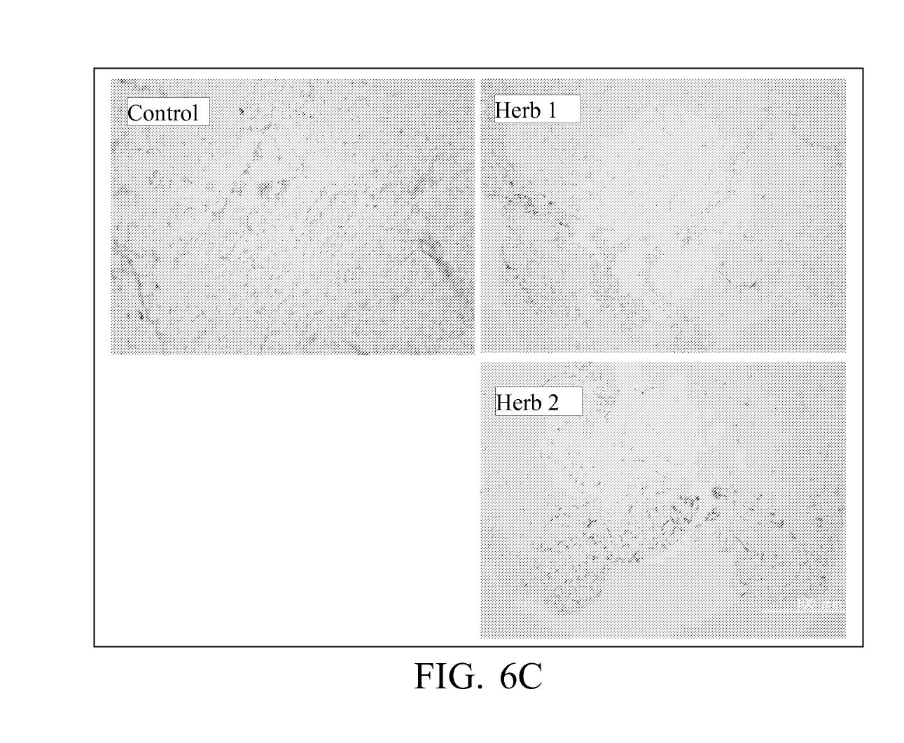

After H&E staining, the Matrigel plugs taken from subcutaneous tissues of mice were observed by a microscope at 200× magnification, and the results are shown in FIG. 6A. According to FIG. 6A, it is shown that human umbilical vein endothelial cells of the Matrigel plugs of the treatment group of extract of whole plants of Sambucus formosana Nakai (Herb 1) has a trend of increasing as compared with the control group. The results of cell counting performed on the 2 observed regions randomly selected under 200× flied of view of each stained sample are shown in FIG. 6B. FIG. 6B shows that the cell number of the human umbilical vein endothelial cells of the control group was 67.63±8.14 cells/region, the cell number of the human umbilical vein endothelial cells of the treatment group of extract of whole plants of Sambucus formosana Nakai (Herb 1) was 99.00±18.63 cells/region, and the cell number of the human umbilical vein endothelial cells of the treatment group of extract of the extract of roots of Isatis indigotica Fort. (Herb 2) was 73.25±10.51 cells/region. After comparing cell density of each treatment group with that of the control group, it is shown that the extract of whole plants of Sambucus formosana Nakai (Herb 1) has more effect of promoting proliferation of human umbilical vein endothelial cells. Through CD31 immunohistochemical staining, it is shown that vein endothelial cells expressing CD31 of the Matrigel plugs of the treatment groups of the extracts of roots of Isatis indigotica Fort. (Herb 2) are all more apparent than that of the control group, as shown in FIG. 6C, and that shows the extracts of roots of Isatis indigotica Fort. (Herb 2) all have the effect of promoting angiogenesis.

### Example 9

### Animal Experiment for Promoting Healing of Diabetic Wounds by Plant Extract

9.1 Method for Healing Diabetic Wounds In Vivo

Diabetic mice were provided by National Laboratory Animal Center, R.O.C, which were BKS.Cg-Dock^{m}+/+Lepr^{db}/JNarl gene transplanted fat mice with type 2 diabetes (called for short to db/db mice). Experimentation for healing diabetic wounds was performed to the mice with the plant extracts mentioned above.

A part of skin (containing the epidermis, the derma, subcutaneous tissues and muscle) of the db/db mouse was cut off, and the size of the cut wound was a circular area with a diameter of 1 cm. Then, a film Tegarderm (3M Healthcare) which is waterproof and allowed ventilation was covered on the wound, and 0.1 mg of the extract of whole plants of Sambucus formosana Nakai and 0.1 mg of the extracts of roots of Isatis indigotica Fort. were mixed well and injected between the film and the wound once every day, 5 days a week until the wound completely healed. The wound was photographed and recorded during the experiment. The area of the wound was analyzed by ImageJ and the statistics were analyzed by student t test.

9.2 Results for Healing Diabetic Wounds In Vivo

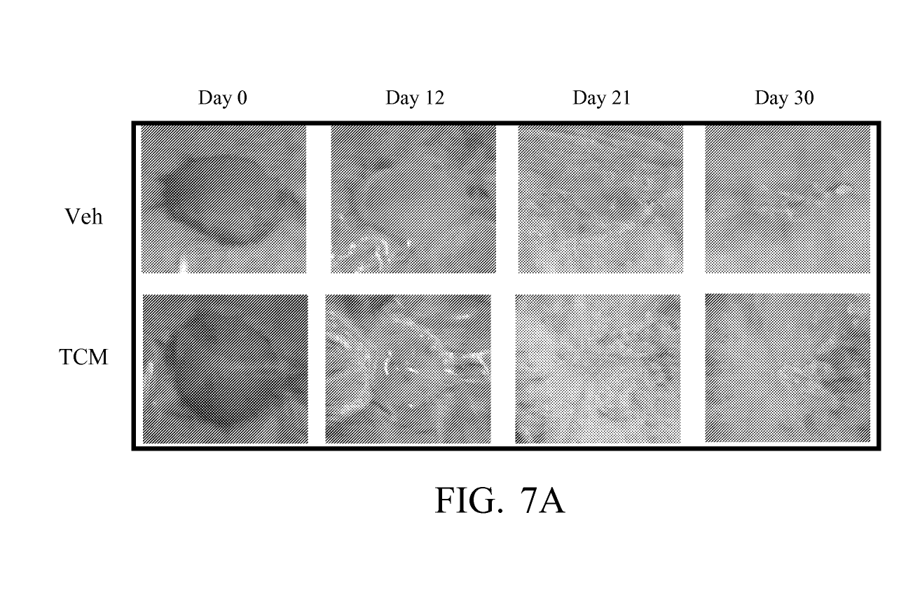

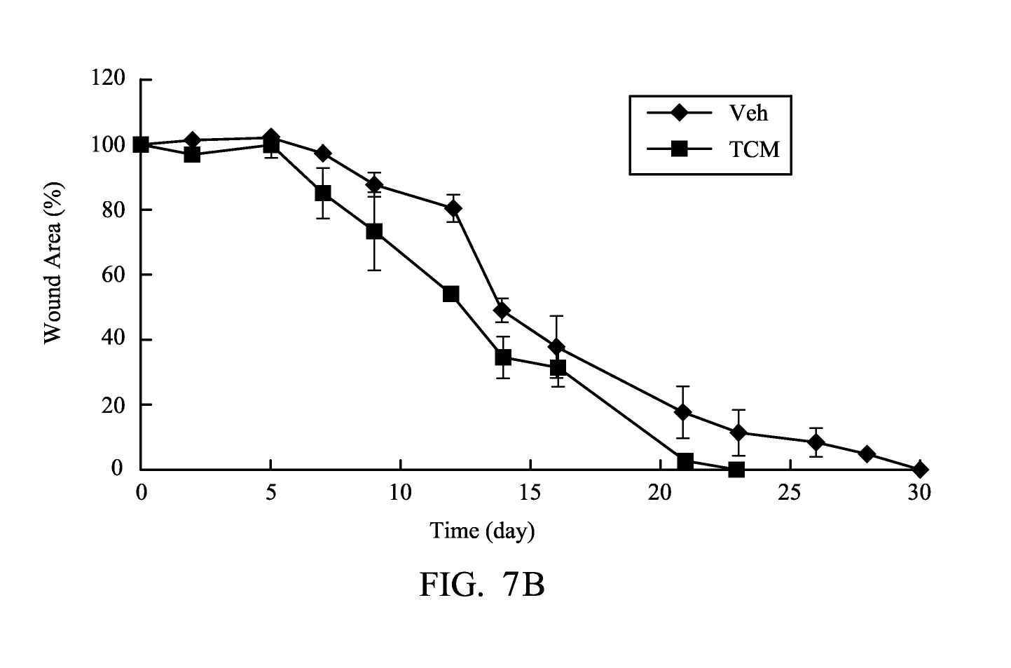

The results of the foregoing experiment are shown in FIG. 7A and FIG. 7B. The wound photographs of day 0 (the day of performing the surgical operation for cutting a part of skin off), day 12, day 21 and day 30 were compared and found that on day 12 the wound of the administration group healed faster than the wound of the control group did. In addition, on day 21, the wound of the administration group healed completely, while that of the control group did not completely heal. Until day 30, the wound of the control group just healed completely. As the wound area of day 0 is considered as 100%, on day 12, the wound area of the administration group was 53.84±2.04%, and the wound area of the control group was 79.66±5.03%; p value<0.05:* (FIG. 7B). According to the results of the animal experiment, it is shown that the mixture of the extract of Sambucus formosana Nakai and the extracts of Isatis indigotica Fort. is capable of promoting healing diabetic wounds significantly to accelerate healing of the wounds of fat mice with type 2 diabetes and reduce the wound area, and to advance the time of complete healing.

It will be apparent to those skilled in the art that various modifications and variations can be made to the disclosed embodiments. It is intended that the specification and examples be considered as exemplary only, with the true scope of the disclosure being indicated by the following claims and their equivalents.

What is claimed is:

1. A pharmaceutical composition, comprising:
an effective amount of a Sambucus plant as an active ingredient for promoting wound healing; and
a pharmaceutically acceptable carrier or medium.

2. The pharmaceutical composition as claimed in claim 1, wherein a part of the Sambucus plant used in the pharmaceutical composition comprises a whole plant of the Sambucus plant, a root of the Sambucus plant, a stem of the Sambucus plant, a leaf of the Sambucus plant, a flower of the Sambucus plant and/or a fruit of the Sambucus plant.

3. The pharmaceutical composition as claimed in claim 1, wherein the Sambucus plant is subjected to an extraction process to yield an extract of the Sambucus plant.

4. The pharmaceutical composition as claimed in claim 3, wherein a part of the Sambucus plant used in the pharmaceutical composition comprises a whole plant of the Sambucus plant, a root of the Sambucus plant, a stem of the Sambucus plant, a leaf of the Sambucus plant, a flower of the Sambucus plant and/or a fruit of the Sambucus plant.

5. The pharmaceutical composition as claimed in claim 1, wherein the Sambucus plant comprises Sambucus formosana Nakai, Sambucus japonica Thunb., Sambucus nigra L., Sambucus williamsii Hance or Sambucus williamsii Hance var. miquelii (Nakai) Y. C. Tang.

6. The pharmaceutical composition as claimed in claim 1, wherein the Sambucus plant is Sambucus formosana Nakai.

7. The pharmaceutical composition as claimed in claim 6, wherein a part of the Sambucus formosana Nakai used in the pharmaceutical composition comprises a whole plant of the Sambucus formosana Nakai, a root of the Sambucus formosana Nakai, a stem of the Sambucus formosana Nakai, a leaf of the Sambucus formosana Nakai, a flower of the Sambucus formosana Nakai and/or a fruit of the Sambucus formosana Nakai.

8. The pharmaceutical composition as claimed in claim 6, wherein the Sambucus formosana Nakai is subjected to an extraction process to yield an extract of the Sambucus formosana Nakai.

9. The pharmaceutical composition as claimed in claim 8, wherein a part of the Sambucus formosana Nakai used in the pharmaceutical composition comprises a whole plant of the Sambucus formosana Nakai, a root of the Sambucus formosana Nakai, a stem of the Sambucus formosana Nakai, a leaf of the Sambucus formosana Nakai, a flower of the Sambucus formosana Nakai and/or a fruit of the Sambucus formosana Nakai.

10. The pharmaceutical composition as claimed in claim 8, wherein an extraction solvent used in the extraction process comprises water, ethanol and/or methanol.

11. The pharmaceutical composition as claimed in claim 6, wherein the sequence of an internal transcribed spacer of ribosomal DNA of Sambucus formosana Nakai is SEQ ID NO: 1 or a sequence with at least 80% sequence identity to SEQ ID NO: 1.

12. The pharmaceutical composition as claimed in claim 6, wherein the sequence of an intergenic non-coding region of chloroplast DNA of Sambucus formosana Nakai, trnH-psbA, is SEQ ID NO: 5 or a sequence with at least 80% sequence identity to SEQ ID NO: 5.

13. The pharmaceutical composition as claimed in claim 1, further comprising an Isatis plant.

14. The pharmaceutical composition as claimed in claim 13, wherein a part of the Isatis plant used in the pharmaceutical composition comprises a whole plant of the Isatis plant, a root of the Isatis plant, a stem of the Isatis plant, a leaf of the Isatis plant, a flower of the Isatis plant and/or a fruit of the Isatis plant.

15. The pharmaceutical composition as claimed in claim 13, wherein the Isatis plant is subjected to an extraction process to yield an extract of the Isatis plant.

16. The pharmaceutical composition as claimed in claim 15, wherein a part of the Isatis plant used in the pharmaceutical composition comprises a whole plant of the Isatis plant, a root of the Isatis plant, a stem of the Isatis plant, a leaf of the Isatis plant, a flower of the Isatis plant and/or a fruit of the Isatis plant.

17. The pharmaceutical composition as claimed in claim 13, wherein the Isatis plant comprises Isatis indigotica Fort., Isatis oblongata DC., Isatis violascens, Isatis tinctoria L. var. praecox (Kit.) Koch, Isatis costata, Isatis minima Bunge or Isatis tinctoria L.

18. The pharmaceutical composition as claimed in claim 13, wherein the Isatis plant is Isatis indigotica Fort.

19. The pharmaceutical composition as claimed in claim 18, wherein a part of the Isatis indigotica Fort. used in the pharmaceutical composition comprises a whole plant of the Isatis indigotica Fort., a root of the Isatis indigotica Fort., a stem of the Isatis indigotica Fort. and/or a leaf of the Isatis indigotica Fort.

20. The pharmaceutical composition as claimed in claim 18, wherein the Isatis indigotica Fort. is subjected to an extraction process to yield an extract of the Isatis indigotica Fort.

21. The pharmaceutical composition as claimed in claim 20, wherein a part of the Isatis indigotica Fort. used in the pharmaceutical composition comprises a whole plant of the Isatis indigotica Fort., a root of the Isatis indigotica Fort. and/or a stem of the Isatis indigotica Fort.

22. The pharmaceutical composition as claimed in claim 20, wherein an extraction solvent used in the extraction process comprises water, ethanol and/or methanol.

23. The pharmaceutical composition as claimed in claim 18, wherein the sequence of an internal transcribed spacer of ribosomal DNA of Isatis indigotica Fort. is SEQ ID NO: 2 or a sequence with at least 80% sequence identity to SEQ ID NO: 2.

24. The pharmaceutical composition as claimed in claim 18, wherein the sequence of an intergenic non-coding region of chloroplast DNA of Isatis indigotica Fort., trnF-trnL, is SEQ ID NO: 6 or a sequence with at least 80% sequence identity to SEQ ID NO: 6.

25. A method for preparing a medication for promoting wound healing, comprising:
using a Sambucus plant.

26. The method for preparing a medication for promoting wound healing as claimed in claim 25, wherein the Sambucus plant is Sambucus formosana Nakai.

27. The method for preparing a medication for promoting wound healing as claimed in claim 26, wherein a part of the Sambucus formosana Nakai used in the pharmaceutical composition comprises a whole plant of the Sambucus formosana Nakai, a root of the Sambucus formosana Nakai, a stem of the Sambucus formosana Nakai, a leaf of the Sambucus formosana Nakai, a flower of the Sambucus formosana Nakai and/or a fruit of the Sambucus formosana Nakai.

28. The method for preparing a medication for promoting wound healing as claimed in claim 26, wherein the Sambucus formosana Nakai is subjected to an extraction process to yield an extract of the Sambucus formosana Nakai.

29. A method for preparing a medication for promoting wound healing as claimed in claim 28, wherein a part of the Sambucus formosana Nakai used in the pharmaceutical composition comprises a whole plant of the Sambucus formosana Nakai, a root of the Sambucus formosana Nakai, a stem of the Sambucus formosana Nakai, a leaf of the Sambucus formosana Nakai, a flower of the Sambucus formosana Nakai and/or a fruit of the Sambucus formosana Nakai.

30. A pharmaceutical composition, comprising:
an effective amount of an Isatis plant as an active ingredient for promoting wound healing; and
a pharmaceutically acceptable carrier or medium.

31. The pharmaceutical composition as claimed in claim 30, wherein a part of the Isatis plant used in the pharmaceutical composition comprises a whole plant of the Isatis plant, a root of the Isatis plant, a stem of the Isatis plant, a leaf of the Isatis plant, a flower of the Isatis plant and/or a fruit of the Isatis plant.

32. The pharmaceutical composition as claimed in claim 30, wherein the Isatis plant is subjected to an extraction process to yield an extract of the Isatis plant.

33. The pharmaceutical composition as claimed in claim 32, wherein a part of the Isatis plant used in the pharmaceutical composition comprises a whole plant of the Isatis plant, a root of the Isatis plant, a stem of the Isatis plant, a leaf of the Isatis plant, a flower of the Isatis plant and/or a fruit of the Isatis plant.

34. The pharmaceutical composition as claimed in claim 30, wherein the Isatis plant comprises Isatis indigotica Fort., Isatis oblongata DC., Isatis violascens, Isatis tinctoria L. var. praecox (Kit.) Koch, Isatis costata, Isatis minima Bunge or Isatis tinctoria L.

35. The pharmaceutical composition as claimed in claim 30, wherein the Isatis plant is Isatis indigotica Fort.

36. The pharmaceutical composition as claimed in claim 35, wherein a part of the Isatis indigotica Fort. used in the pharmaceutical composition comprises a whole plant of the Isatis indigotica Fort., a root of the Isatis indigotica Fort., a stem of the Isatis indigotica Fort. and/or a leaf of the Isatis indigotica Fort.

37. The pharmaceutical composition as claimed in claim 35, wherein the Isatis indigotica Fort. is subjected to an extraction process to yield an extract of the Isatis indigotica Fort.

38. The pharmaceutical composition as claimed in claim 37, wherein a part of the Isatis indigotica Fort. used in the pharmaceutical composition comprises a whole plant of the Isatis indigotica Fort., a root of the Isatis indigotica Fort. and/or a stem of the Isatis indigotica Fort.

39. The pharmaceutical composition as claimed in claim 37, wherein an extraction solvent used in the extraction process comprises water, ethanol and/or methanol.

40. The pharmaceutical composition as claimed in claim 35, wherein the sequence of an internal transcribed spacer of ribosomal DNA of Isatis indigotica Fort. is SEQ ID NO: 2 or a sequence with at least 80% sequence identity to SEQ ID NO: 2.

41. The pharmaceutical composition as claimed in claim 35, wherein the sequence of an intergenic non-coding region of chloroplast DNA of Isatis indigotica Fort., trnF-trnL, is SEQ ID NO: 6 or a sequence with at least 80% sequence identity to SEQ ID NO: 6.

42. A method for preparing a medication for promoting wound healing, comprising:
using an Isatis plant.

43. The method for preparing a medication for promoting wound healing as claimed in claim 42, wherein the Isatis plant is Isatis indigotica Fort.

44. The method for preparing a medication for promoting wound healing as claimed in claim 43, wherein a part of the Isatis indigotica Fort. used in the pharmaceutical composition comprises a whole plant of the Isatis indigotica Fort., a root of the Isatis indigotica Fort., a stem of the Isatis indigotica Fort. and/or a leaf of the Isatis indigotica Fort.

45. The method for preparing a medication for promoting wound healing as claimed in claim 43, wherein the Isatis indigotica Fort. is subjected to an extraction process to yield an extract of the Isatis indigotica Fort.

46. A method for preparing a medication for promoting wound healing as claimed in claim 45, wherein a part of the Isatis indigotica Fort. used in the pharmaceutical composition comprises a whole plant of the Isatis indigotica Fort., a root of the Isatis indigotica Fort., a stem of the Isatis indigotica Fort. and/or a leaf of the Isatis indigotica Fort.


SEQUENCE LISTING (10 sequences)

SEQ ID NO: 1 - DNA, 622 bp, Sambucus formosana Nakai
tcgatacctttatcgtaaacagaacgacccgcgaacgattgatcatcactctcggtgggctggtgtcttagctgattccgtgcctgccgattccgtggttatgcgcgtggtctcagccaagattcatatctcggttgggtcatgcgcctagcttccggatatcaccaaaccccggcacgaaaagtgtcaaggaacatgcaactaaacagcctgcgttcgcctacccggagacggtgtttgcgtggacgctgtgctgcaatctaaagtctaaaacgactctcggcaacggatatctcggctctcgcatcgatgaagaacgtagcgaaatgcgatacttggtgtgaattgcagaatcccgtgaaccatcgagtctttgaacgcaagttgcgccctaagccttctggccgagggcacgtctgcctgggtgtcacaaatcgtcgtccccccatcctctcgaggataatggacggaagctggtctcccgtgtgttaccgcacgcggttggccaaaatccgagctaaggacgcaaggagcgtctcgacatgcggtggtgaattaaaacctcgtcataccgttggccgctcctgtcctgatgctctcgatgacccaaagtcctcaacgc

SEQ ID NO: 2 - DNA, 753 bp, Isatis indigotica Fort.
atggtccggtgagtgttcggatcgcggcgacgtgggcggttcgctgccggcgacgtcgcgagaagtccactgaaccttatcatttagaggaaggagaagtcgtaacaaggtttccgtaggtgaacctgcggaaggatcattgtcgaaacctgcacagcagaatgacccgcgaactcgttttcatattggggctcgtcgtcctaggtgcgcaagcgcttggccggcggacttcggtcagggcgccctcggtgtactgaccaaacaacgaaccccggcgcgatctgcgccaaggaatttttactaaagagcgtgtctatcgttgccccgttcgcggtgtgcacggtaggcctgcgcctttgaaaaacaaacgactctcggcaacggatatctcggctctcgcatcgatgaagaacgtagcgaaatgcgatacttggtgtgaattgcagaatcccgtgaaccatcgagtttttgaacgcaagttgcgcccgaagccattaggccgagggcacgtctgcctgggcgtcacacattgccgtcgcccccttccaatttcccattcttggggacgttagtagtcgggcggatattggtctcccgtgctctcgagcgcggttggcccaaaagcgagtccccgacaacggacgtcacgacaagtggtggttgaaaagccttctaatcctgtcgtgcaccaattcgttgtcaggggcatcgagttgaccctgacgcgtcgtctccgacgtcgctccgatcgcg

SEQ ID NO: 3 - DNA, 21 bp, Artificial sequence
  Forward primer
tagaggaaggagaagtcgtaa

SEQ ID NO: 4 - DNA, 21 bp, Artificial sequence
  Reverse primer
gacgcttctccagactacaat

SEQ ID NO: 5 - DNA, 460 bp, Sambucus formosana Nakai
ccctactagtatttagattacaactagtatttacattacaaacattcatcatttttttttattatttctgagatacaaatcttaagtaaagaaaatattttaaaattttctctatttttgattatttaatagaataaaaaagaaaaaacacacaatttcagaaaagattgggaagaggttatcaaaatgctatattaatacagaacaacagcacaatacttaagtatgacccaacccataaaaatccttgtttttttcatttaacgaaaatatgtaagaaaaataaataaaataaaaaaggagcaataccgccctcttgatagaacaagaaagaggttattgctccttttttttatttcaaatactcgtatacactaagaccaggtcttatccatttgtggatggagcttcaagagcagctaggtctagagggaagttgtgagcattacgttcatgcata

SEQ ID NO: 6 - DNA, 791 bp, Isatis indigotica Fort.
tttttatatttatatattaaaattggtacgaggatttcagtcctctgctctaccaactgagctatcccggccattaccgaagtatcatcctcattttactagatgacttaggtctatgtcaattaaaagaaaagaaacgaaaaagtaggaaatgcaaaaagttttggaccgggaatttcttggatctttgaaaagaagactttctaagttttaacatgaaaaatgatatagattctaaataattcaaatcgacatttctttctcatttgtacgtgtatgatatatcccacaagacttgtatataataagaaaagaaattatagtttgtaaaagcgtcggatgaatgaaaaaagataatgcatttttgaataactaaaaaaagggtaaggtgtcaaacggacttttttggggagtagggttggggatagagggacttgaaccctcacgattttaaaagtcaacggattttcatcttactataaatttcattgttgtcagtattgacatgtagaatgggactctatctttattctcgtccgattattccaccaaggatctatcagactatgaagtgaatcatttgatcaacacaaggtagtgaactccatttgttagaacagcttccattgagtctctgcacctatcccttttttctcgttttctaaactctggtttgttcgcgtaacccaggatttggctcaggattgcccagtgttaattccagggtttctctgaatttgaaagttatcacttagtaggtttccataccaaggctcaatccaattaagtcc

SEQ ID NO: 7 - DNA, 20 bp, Artificial sequence
  Forward primer
gctaaccttggtatggaagt

SEQ ID NO: 8 - DNA, 23 bp, Artificial sequence
  Reverse primer
tgatccacttggctacatccgcc

SEQ ID NO: 9 - DNA, 20 bp, Artificial sequence
  Forward primer
cgaaatcggtagacgctacg

SEQ ID NO: 10 - DNA, 20 bp, Artificial sequence
  Reverse primer
atttgaactggtgacacgag